(19) United States
(12) Patent Application Publication
(10) Pub. No.: US 2021/0346303 A1
(43) Pub. Date: Nov. 11, 2021

(54) COATING METHOD

(71) Applicant: SUMITOMO DAINIPPON PHARMA CO., LTD., Osaka-shi (JP)

(72) Inventors: Mitsuaki KOBIKI, Kobe-shi (JP); Takumi ASADA, Suita-shi (JP)

(73) Assignee: SUMITOMO DAINIPPON PHARMA CO., LTD., Osaka-shi (JP)

(21) Appl. No.: 17/286,226

(22) PCT Filed: Oct. 17, 2019
(86) PCT No.: PCT/JP2019/040923

§ 371 Date: Apr. 16, 2021

(30) Foreign Application Priority Data
Oct. 18, 2018 (JP) 2018-196987

(51) Int. Cl. A61K 9/50; A61K 31/423; A61K 31/167; A61K 31/522

(52) U.S. Cl. CPC A61K 9/5026; A61K 31/423; A61K 9/5089; A61K 31/522; A61K 31/167

(57) ABSTRACT

Provided is a method for producing particles coated by a coatable first polymer and a lubricant. A method for producing particles coated by a first polymer and a lubricant, wherein the production method is characterized by: including a step for adding a first polymer and a lubricant to core particles that include a component of interest and a second polymer, and coating while rolling the mixture and spraying a solvent capable of dissolving the first polymer; and the particles coated by the first polymer and lubricant being component of interest-containing hollow particles.

### TECHNICAL FIELD

The present disclosure relates to a technology for strengthening the function of a component of interest-containing particle. The present disclosure also relates to a coating method. The present disclosure relates to a coating method that is efficient and requires a short period of time. In a preferred embodiment, the present disclosure relates to a coated particle having a plurality of functions.

### BACKGROUND ART

A formulation technology generally manufactures component of interest-containing particles by granulating only a component of interest or a component of interest mixed with another formulation component, and then further granulating by mixing another component, mixing another granule, or adding another component to prepare a tablet, a granule, or capsule agent by filling a capsule.

### SUMMARY OF INVENTION

### Technical Problem

A method of dissolving a release controlled macromolecule into a solvent and spraying the solution has a problem in that the ability to control release of coated particles is high, but the coating takes a long period of time, and the production yield is low for each manufacture. It is possible to reduce the coating time or improve the production yield to solve the problem, but this instead results in a problem of reduced ability to control release of coated particles, and difficulty in adjusting the extent of controlled release. In this manner, it was difficult to simultaneously achieve release controlling ability and productivity.

### Solution to Problem

As a result of diligent studies, the inventors completed the present disclosure by finding that coated particles imparted with a function to control a powdered macromolecule can be efficiently manufactured by very simple means of mixing the powdered macromolecule and a lubricant with a nuclear particle comprising a macromolecule, and stirring and granulating while spraying a solvent that can dissolve the powdered macromolecule.

The inventors completed the present disclosure by also finding that coated particles, wherein macromolecule particles are prevented from aggregating with one another and a nuclear particle is imparted with a function to control a powdered macromolecule, can be efficiently manufactured by very simple means of mixing the powdered macromolecule and a lubricant with a nuclear particle comprising a macromolecule, and stirring and granulating while spraying a solvent that can dissolve the powdered macromolecule.

### (Item 1)

A manufacturing method of a particle coated with a first macromolecule and a lubricant, the particle being a component of interest-containing hollow particle comprising a component of interest and a second macromolecule, the method comprising:

adding the first macromolecule and the lubricant to a nuclear particle comprising the component of interest and the second macromolecule and coating the resulting mixture by spraying a solvent that can dissolve the first macromolecule while rolling the mixture.

### (Item 2)

The manufacturing method of item 1, wherein the coated particle comprises an inner core layer comprising the component of interest and the second macromolecule and a coating layer comprising the first macromolecule and the lubricant.

### (Item 3)

The manufacturing method of item 1 or 2, further comprising generating the nuclear particle by mixing the component of interest and the second macromolecule.

### (Item 4)

The manufacturing method of any one of items 1 to 3, wherein a D90 value of a mixture of the first macromolecule and the lubricant is 100 μm or less.

### (Item 5)

The manufacturing method of any one of items 1 to 4, wherein a mean particle size of the first macromolecule and the lubricant is 25 μm or less.

### (Item 6)

The manufacturing method of any one of items 1 to 5, wherein a D100 value of the first macromolecule and the lubricant is 150 μm or less.

### (Item 7)

The manufacturing method of any one of items 1 to 6, wherein all of the first macromolecule and the lubricant pass through a 100 mesh sieve.

### (Item 8)

The manufacturing method of any one of items 1 to 7, wherein the first macromolecule is selected from one or more of enteric soluble macromolecule.

### (Item 9)

The manufacturing method of any one of items 1 to 8, wherein the lubricant is selected from one or more of magnesium aluminosilicate, talc, Red Ferric Oxide, Yellow Ferric Oxide, titanium oxide, sodium stearyl fumarate, and magnesium stearate.

### (Item 10)

The manufacturing method of any one of items 1 to 9, wherein the lubricant is selected from one or more of talc, titanium oxide and sodium stearyl fumarate.

### (Item 11)

The manufacturing method of any one of items 1 to 10, wherein the lubricant is talc.

### (Item 12)

The manufacturing method of any one of items 1 to 11, wherein a weight ratio of the first macromolecule to the lubricant is between 1:10 and 10:1.

### (Item 13)

The manufacturing method of any one of items 1 to 12, wherein the first macromolecule and the lubricant are 10% by weight to 50% by weight with respect to the nuclear particle.

### (Item 14)

The manufacturing method of any one of items 1 to 13, wherein a bulk density of the lubricant is 0.1 g/mL or greater.

### (Item 15)

The manufacturing method of any one of items 1 to 14, wherein a mean molecular weight of the first macromolecule is 1000 to 10000000.

### (Item 16)

The manufacturing method of any one of items 1 to 15, wherein the first macromolecule are a water-insoluble cellulose ether, a water-insoluble acrylic acid copolymer, vinyl acetate resin, or a combination thereof.

### (Item 17)

The manufacturing method of any one of items 1 to 16, wherein the second macromolecule is the same as the first macromolecule.

### (Item 18)

The manufacturing method of any one of items 1 to 17, wherein the component of interest is a drug, a quasi-drug, a cosmetic, an agricultural chemical, a supplement, or a food product.

### (Item 19)

A composition for imparting a function of a macromolecule to a component of interest-containing hollow particle consisting of a shell and a hollow section, comprising the macromolecule and a lubricant.

### (Item 20)

A composition comprising a first macromolecule and a lubricant for imparting a function of the first macromolecule to a component of interest-containing hollow particle consisting of a shell and a hollow section, wherein the component of interest-containing hollow particle comprises a second macromolecule and a component of interest.

### (Item 21)

A composition comprising a lubricant for imparting a function of a first macromolecule to a component of interest-containing hollow particle consisting of a shell and a hollow section, wherein the component of interest-containing hollow particle comprises a second macromolecule and a component of interest, and the first macromolecule is provided with the lubricant.

### (Item 22)

The composition of any one of items 19 to 21, wherein the function comprises sustained release, enteric solubility, stomach solubility, bitterness masking, or photostability.

### (Item 23)

The composition of any one of items 19 to 22, wherein the function is enteric solubility.

### (Item 24)

A composition comprising a first macromolecule and a lubricant for imparting a function of the lubricant to a component of interest-containing hollow particle consisting of a shell and a hollow section, wherein the component of interest-containing hollow particle comprises a second macromolecule and a component of interest.

### (Item 25)

A composition comprising a first macromolecule for imparting a function of a lubricant to a component of interest-containing hollow particle consisting of a shell and a hollow section, wherein the component of interest-containing hollow particle comprises a second macromolecule and a component of interest.

### (Item 26)

The composition of item 23 or 24, wherein the function comprises bitterness masking or photostability.

### (Item 27)

The composition of any one of items 19 to 26, wherein a D90 value of a mixture of the macromolecule and the lubricant is 100 μm or less.

### (Item 28)

The composition of any one of items 19 to 27, wherein a mean particle size of a mixture of the macromolecule and the lubricant is 25 μm or less.

### (Item 29)

The composition of any one of items 19 to 28, wherein a D100 value of a mixture of the macromolecule and the lubricant is 150 μm or less.

### (Item 30)

The composition of any one of items 19 to 29, wherein all of a mixture of the macromolecule and the lubricant pass through a 100 mesh sieve.

### (Item 31)

The composition of any one of items 19 to 30, wherein the macromolecule is selected from one or more of enteric soluble macromolecules.

### (Item 32)

The composition of any one of items 19 to 31, wherein the lubricant is selected from one or more of magnesium aluminosilicate, talc, Red Ferric Oxide, Yellow Ferric Oxide, titanium oxide, sodium stearyl fumarate, and magnesium stearate.

### (Item 33)

The composition of any one of items 19 to 32, wherein the lubricant is selected from one or more of talc, titanium oxide, and sodium stearyl fumarate.

### (Item 34)

The composition of any one of items 19 to 33, wherein the lubricant is talc.

### (Item 35)

The composition of any one of items 19 to 34, wherein the component of interest is a drug, a quasi-drug, a cosmetic, an agricultural chemical, a supplement, or a food product.

### (Item 36)

A particle consisting of a shell and a hollow section, coated with a first macromolecule and a lubricant, wherein the particle comprises a second macromolecule, and a property of the first macromolecule and/or the second macromolecule is more enhanced relative to the particle in the absence of the lubricant.

### (Item 37)

The particle of item 36, wherein the first macromolecule is the same as the second macromolecule.

### (Item 38)

The particle of item 36 or 37, wherein a D90 value of a mixture of the first macromolecule and the lubricant is 100 μm or less.

### (Item 39)

The particle of any one of items 36 to 38, wherein a mean particle size of a mixture of the first macromolecule and the lubricant is 25 μm or less.

### (Item 40)

The particle of any one of items 36 to 39, wherein a D100 value of a mixture of the first macromolecule and the lubricant is 150 μm or less.

### (Item 41)

The particle of any one of items 36 to 40, wherein all of a mixture of the first macromolecule and the lubricant pass through a 100 mesh sieve.

### (Item 42)

The particle of any one of items 36 to 41, wherein the first macromolecule is selected from one or more of enteric soluble macromolecules.

### (Item 43)

The particle of any one of items 36 to 42, wherein the lubricant is selected from one or more of magnesium aluminosilicate, talc, Red Ferric Oxide, Yellow Ferric Oxide, titanium oxide, sodium stearyl fumarate, and magnesium stearate.

### (Item 44)

The particle of any one of items 36 to 43, wherein the lubricant is selected from one or more of talc, titanium oxide, and sodium stearyl fumarate.

### (Item 45)

The particle of any one of items 36 to 44, wherein the lubricant is talc.

### (Item 1a)

A manufacturing method of a particle coated with first macromolecule and lubricant, the particle being a component of interest-containing hollow particle comprising a component of interest and a second macromolecule, the method comprising:

adding the first macromolecule and the lubricant to a nuclear particle comprising the component of interest and the second macromolecule and coating the resulting mixture by spraying a solvent that can dissolve the first macromolecule while rolling the mixture.

### (Item 2a)

The manufacturing method of item 1a, wherein the coated particle comprises an inner core layer comprising the component of interest and the second macromolecule and a coating layer comprising the first macromolecule and the lubricant.

### (Item 3a)

The manufacturing method of item 1a or 2a, further comprising generating the nuclear particle by mixing the component of interest and the second macromolecule.

### (Item 4a)

The manufacturing method of any one of items 1a to 3a, wherein a D90 value of the first macromolecule and the lubricant is 100 μm or less.

### (Item 5a)

The manufacturing method of any one of items 1a to 4a, wherein a mean particle size of the first macromolecule and the lubricant is 25 μm or less.

### (Item 6a)

The manufacturing method of any one of items 1a to 5a, wherein a D100 value of the first macromolecule and the lubricant is 150 μm or less.

(Item 6a-1)

The manufacturing method of any one of items 1a to 5a, wherein a D99 value of the first macromolecule and the lubricant is 150 μm or less.

### (Item 7a)

The manufacturing method of any one of items 1a to 6a and 6a-1, wherein the first macromolecule and the lubricant pass through a 100 mesh sieve.

(Item 7a-1)

The manufacturing method of any one of items 1a to 7a, wherein the first macromolecule are selected from one or more of a water-soluble macromolecule, a water-insoluble macromolecule, an enteric soluble macromolecule, and a stomach soluble macromolecule.

(Item 7a-2)

The manufacturing method of any one of items 1a to 7a-1, wherein the first macromolecule is a water-soluble macromolecule.

(Item 7a-3)

The manufacturing method of any one of items 1a to 7a-1, wherein the first macromolecule is a water-insoluble macromolecule.

### (Item 8a)

The manufacturing method of any one of items 1a to 7a-1, wherein the first macromolecule is an enteric soluble macromolecule.

(Item 8a-1)

The manufacturing method of any one of items 1a to 7a-1, wherein the first macromolecule is a stomach soluble macromolecule.

### (Item 9a)

The manufacturing method of any one of items 1a to 8a and 8a-1, wherein the lubricant is selected from one or more of magnesium aluminosilicate, talc, Red Ferric Oxide, Yellow Ferric Oxide, titanium oxide, sodium stearyl fumarate, and magnesium stearate.

### (Item 10a)

The manufacturing method of any one of items 1a to 9a, wherein the lubricant is selected from one or more of talc, titanium oxide, and sodium stearyl fumarate.

### (Item 11a)

The manufacturing method of any one of items 1a to 10a, wherein the lubricant is talc.

### (Item 12a)

The manufacturing method of any one of items 1a to 11a, wherein a weight ratio of the first macromolecule to the lubricant is between 1:5 and 5:1.

### (Item 13a)

The manufacturing method of any one of items 1a to 12a, wherein the first macromolecule and the lubricant are 10% by weight to 100% by weight with respect to the nuclear particle.

### (Item 14a)

The manufacturing method of any one of items 1a to 13a, wherein a bulk density of the lubricant is 0.1 g/mL or greater.

### (Item 15a)

The manufacturing method of any one of items 1a to 14a, wherein a mean molecular weight of the first macromolecule is 1000 to 10000000.

### (Item 16a)

The manufacturing method of any one of items 1a to 15a, wherein the first macromolecule is a water-insoluble cellulose ether, a water-insoluble acrylic acid copolymer, vinyl acetate resin, or a combination thereof.

### (Item 17a)

The manufacturing method of any one of items 1a to 16a, wherein the second macromolecule is the same as the first macromolecule.

### (Item 18a)

The manufacturing method of any one of items 1a to 17a, wherein the component of interest is a drug, a quasi-drug, a cosmetic, an agricultural chemical, a supplement, or a food product.

### (Item 19a)

A composition for imparting a function of a first macromolecule to a component of interest-containing hollow particle consisting of a shell and a hollow section, comprising the macromolecule and a lubricant.

### (Item 20a)

A composition comprising a first macromolecule and a lubricant for imparting a function of the first macromolecule to a component of interest-containing hollow particle consisting of a shell and a hollow section, wherein the component of interest-containing hollow particle comprises a second macromolecule and a component of interest.

### (Item 21a)

A composition comprising a lubricant for imparting a function of a first macromolecule to a component of interest-containing hollow particle consisting of a shell and a hollow section, wherein the component of interest-containing hollow particle comprises a second macromolecule and a component of interest, and the first macromolecule is provided with the lubricant.

### (Item 22a)

The composition of any one of items 19a to 21a, wherein the function comprises fast release, sustained release, enteric solubility, stomach solubility, bitterness masking, or photostability.

(Item 22a-1)

The composition of any one of items 19a to 22a, wherein the function is fast release.

(Item 22a-2)

The composition of any one of items 19a to 22a, wherein the function is sustained release.

### (Item 23a)

The composition of any one of items 19a to 22a, wherein the function is enteric solubility.

(Item 23a-1)

The composition of any one of items 19a to 22a, wherein the function is stomach solubility.

### (Item 24a)

A composition comprising a first macromolecule and a lubricant for imparting a function of the lubricant to a component of interest-containing hollow particle consisting of a shell and a hollow section, wherein the component of interest-containing hollow particle comprises a second macromolecule and a component of interest.

### (Item 25a)

A composition comprising a first macromolecule for imparting a function of a lubricant to a component of interest-containing hollow particle consisting of a shell and a hollow section, wherein the component of interest-containing hollow particle comprises a second macromolecule and a component of interest.

### (Item 26a)

The composition of any one of items 19a to 25a, wherein the function comprises bitterness masking or photostability.

### (Item 27a)

The composition of any one of items 19a to 26a, wherein a D90 value of a mixture of the first macromolecule and the lubricant is 100 μm or less.

### (Item 28a)

The composition of any one of items 19a to 27a, wherein a mean particle size of a mixture of the first macromolecule and the lubricant is 25 μm or less.

### (Item 29a)

The composition of any one of items 19a to 28a, wherein a D100 value of a mixture of the first macromolecule and the lubricant is 150 μm or less.

(Item 29a-1)

The composition of any one of items 19a to 28a, wherein a D99 value of a mixture of the first macromolecule and the lubricant is 150 μm or less.

### (Item 30a)

The composition of any one of items 19a to 29a, wherein all of a mixture of the first macromolecule and the lubricant pass through a 100 mesh sieve.

### (Item 31a)

The composition of any one of items 19a to 30a, wherein the first macromolecule is selected from one or more of a water-soluble macromolecule, a water-insoluble macromolecule, an enteric soluble macromolecule, and a stomach soluble macromolecule.

(Item 31a-1)

The composition of any one of items 19a to 31a, wherein the first macromolecule is a water-soluble macromolecule.

(Item 31a-2)

The composition of any one of items 19a to 31a, wherein the first macromolecule is a water-insoluble macromolecule.

(Item 31a-3)

The composition of any one of items 19a to 31a, wherein the first macromolecule is an enteric soluble macromolecule.

(Item 31a-4)

The composition of any one of items 19a to 31a, wherein the first macromolecule is a stomach soluble macromolecule.

### (Item 32a)

The composition of any one of items 19a to 31a and 31a-1 to 31a-4, wherein the lubricant is selected from one or more of magnesium aluminosilicate, talc, Red Ferric Oxide, Yellow Ferric Oxide, titanium oxide, sodium stearyl fumarate, and magnesium stearate.

### (Item 33a)

The composition of any one of items 19a to 32a, wherein the lubricant is selected from one or more of talc, titanium oxide, and sodium stearyl fumarate.

### (Item 34a)

The composition of any one of items 19a to 33a, wherein the lubricant is talc.

### (Item 35a)

The composition of any one of items 19a to 34a, wherein the component of interest is a drug, a quasi-drug, a cosmetic, an agricultural chemical, a supplement, or a food product.

### (Item 36a)

A particle consisting of a shell and a hollow section, coated with a first macromolecule and a lubricant, wherein the particle comprises a second macromolecule, and a property of the first macromolecule and/or the second macromolecule is more enhanced relative to the particle in the absence of the lubricant.

(Item 36a-1)

A particle consisting of a shell and a hollow section, coated with a first macromolecule and a lubricant, wherein the particle comprises a second macromolecule, and comprises different properties, which are a property of the first macromolecule and a property of the second macromolecule.

(Item 36a-2)

The particle of item 36a-1, wherein the different properties are selected from two or more of fast release, sustained release, enteric solubility, stomach solubility, bitterness masking, and photostability.

(Item 36a-3)

The particle of item 36a-2, wherein the properties comprise fast release.

(Item 36a-4)

The particle of item 36a-2, wherein the properties comprise sustained release.

(Item 36a-5)

The particle of item 36a-2, wherein the properties comprise enteric solubility.

(Item 36a-6)

The particle of item 36a-2, wherein the properties comprise stomach solubility.

(Item 36a-7)

The particle of item 36a-2, wherein the properties comprise bitterness masking.

(Item 36a-8)

The particle of item 36a-2, wherein the properties comprise photostability.

### (Item 37a)

The particle of any one of items 36a to 36a-8, wherein the first macromolecule is the same as the second macromolecule.

### (Item 38a)

The particle of any one of items 36a to 37a, wherein a D90 value of a mixture of the first macromolecule and the lubricant is 100 μm or less.

### (Item 39a)

The particle of any one of items 36a to 38a, wherein a mean particle size of a mixture of the first macromolecule and the lubricant is 25 μm or less.

### (Item 40a)

The particle of any one of items 36a to 39a, wherein a D100 value of a mixture of the first macromolecule and the lubricant is 150 μm or less.

(Item 40a-1)

The particle of any one of items 36a to 39a, wherein a D99 value of a mixture of the first macromolecule and the lubricant is 150 μm or less.

### (Item 41a)

The particle of any one of items 36a to 40a, wherein all of a mixture of the first macromolecule and the lubricant pass through a 100 mesh sieve.

### (Item 42a)

The particle of any one of items 36a to 41a, wherein the first macromolecule is selected from one or more of a water-soluble macromolecule, a water-insoluble macromolecule, an enteric soluble macromolecule, and a stomach soluble macromolecule.

(Item 42a-1)

The particle of any one of items 36a to 42a, wherein the first macromolecule is a water-soluble macromolecule.

(Item 42a-2)

The particle of any one of items 36a to 42a, wherein the first macromolecule is a water-insoluble macromolecule.

(Item 42a-3)

The particle of any one of items 36a to 42a, wherein the first macromolecule is an enteric soluble macromolecule.

(Item 42a-4)

The particle of any one of items 36a to 42a, wherein the first macromolecule is a stomach soluble macromolecule.

### (Item 43a)

The particle of any one of items 36a to 42a and 42a-1 to 42a-4, wherein the lubricant is selected from one or more of magnesium aluminosilicate, talc, Red Ferric Oxide, Yellow Ferric Oxide, titanium oxide, sodium stearyl fumarate, and magnesium stearate.

### (Item 44a)

The particle of any one of items 36a to 43a, wherein the lubricant is selected from one or more of talc, titanium oxide, and sodium stearyl fumarate.

### (Item 45a)

The particle of any one of items 36a to 44a, wherein the lubricant is talc.

The present disclosure is intended so that one or more of the above features can be provided as the explicitly disclosed combinations as well as other combinations thereof. Additional embodiments and advantages of the present disclosure are recognized by those skilled in the art by reading and understanding the following detailed description as needed.

### Advantageous Effects of Invention

The present disclosure provides a coating method that is efficient and requires a short period of time. The present disclosure also provides a method that improves the coatability (coating time and coverage). The method of the present disclosure further provides a component of interest-containing hollow particle using a hollow particle for a nuclear particle provided by the method of the present disclosure.

The component of interest-containing hollow particle of the present disclosure can perform complex release control by coating a macromolecule with a controlling ability that is different from a polymer release controlling ability in a nuclear particle. Specifically, a particle with a complex release control ability, which is configured to not release a component of interest in the stomach, but sustainably release the component of interest in the intestines, can be readily manufactured by coating a particle having a sustained release function in a nuclear particle with a macromolecule having an enteric soluble function. A plurality of desired functionalities (e.g., fast release, enteric solubility, stomach solubility, sustained release, bitterness masking, photostability, or the like) can be imparted by selecting the type of coating macromolecule, macromolecule contained in a nuclear particle, and a lubricant, which allows a formulation that attains a desired efficacy by having a component of interest absorbed at a desired site at a desired time to be provided. Furthermore, the particle size and particle size distribution of component of interest-containing hollow particles can be controlled in any manner by selecting the particle size and particle size distribution of nuclear particles, so that particles matching the objective can be readily manufactured.

### BRIEF DESCRIPTION OF DRAWINGS

FIG. 1A shows the appearance of nuclear particles in Comparative Example 1.

FIG. 1B shows the appearance of nuclear particles in Comparative Example 1.

FIG. 2A shows the appearance of coated particles in Example 1-1.

FIG. 2B shows the appearance of coated particles in Example 1-1.

FIG. 3 shows results of dissolution tests on a 1st fluid in the Japanese Pharmacopoeia in Comparative Example 1 and Examples 1-1 and 1-2.

FIG. 4 shows results of dissolution tests on a 2nd fluid in the Japanese Pharmacopoeia in Comparative Example 1 and Example 1-2.

FIG. 5 shows results of dissolution tests on a 1st fluid in the Japanese Pharmacopoeia in Comparative Example 1 and Examples 2-1 and 2-2.

FIG. 6 shows results of dissolution tests on a 2nd fluid in the Japanese Pharmacopoeia in Comparative Example 1 and Example 2-2.

FIG. 7 shows results of dissolution tests on a 1st fluid in the Japanese Pharmacopoeia in Comparative Example 1 and Examples 3-1 to 3-4.

FIG. 8 shows results of dissolution tests on a 2nd fluid in the Japanese Pharmacopoeia in Comparative Example 1 and Examples 3-2 and 3-4.

FIG. 9 shows results of dissolution tests on a 1st fluid in the Japanese Pharmacopoeia in Comparative Example 1 and Examples 1-2, 4-2, and 4-4.

FIG. 10 shows results of dissolution tests on a 2nd fluid in the Japanese Pharmacopoeia in Comparative Example 1 and Examples 1-2, 4-2, and 4-4.

FIG. 11 shows results of dissolution tests on a 1st fluid for dissolution test in the Japanese Pharmacopoeia in Comparative Example 5 and Examples 5-1 and 5-2.

FIG. 12 shows results of dissolution tests on a 2nd fluid for dissolution test in the Japanese Pharmacopoeia in Comparative Example 5 and Examples 5-1 and 5-2.

FIG. 13 shows results of dissolution tests on a 1st fluid for dissolution test in the Japanese Pharmacopoeia in Comparative Example 6 and Examples 6-1 and 6-2.

FIG. 14 shows results of dissolution tests on a 2nd fluid for dissolution test in the Japanese Pharmacopoeia in Comparative Example 6 and Examples 6-1 and 6-2.

FIG. 15 shows results of dissolution tests on a 1st fluid for dissolution test in the Japanese Pharmacopoeia in Comparative Example 7 and Examples 7-1 and 7-2.

FIG. 16 shows results of dissolution tests on a 2nd fluid for dissolution test in the Japanese Pharmacopoeia in Comparative Example 7 and Examples 7-1 and 7-2.

FIG. 17 shows results of dissolution tests on a 1st fluid for dissolution test in the Japanese Pharmacopoeia in Comparative Example 8 and Examples 8-1 and 8-2.

FIG. 18 shows results of dissolution tests on a 2nd fluid for dissolution test in the Japanese Pharmacopoeia in Comparative Example 8 and Examples 8-1 and 8-2.

### DESCRIPTION OF EMBODIMENTS

The present disclosure is described in detail hereinafter. Throughout the entire specification, a singular expression should be understood as encompassing the concept thereof in plural form, unless specifically noted otherwise. Thus, singular articles (e.g., “a”, “an”, “the”, and the like in the case of English) should also be understood as encompassing the concept thereof in plural form, unless specifically noted otherwise. Further, the terms used herein should be understood to be used in the meaning that is commonly used in the art, unless specifically noted otherwise. Thus, unless defined otherwise, all terminologies and scientific technical terms that are used herein have the same meaning as the general understanding of those skilled in the art to which the present disclosure pertains. In case of a contradiction, the present specification (including the definitions) takes precedence.

A preferred embodiment of each definition can be combined with a preferred embodiment of another definition, or incorporated into a corresponding definition specified in items 1 to 45 herein.

As used herein, “mean particle size” refers to cumulative 50% point of particle size (D50) in volume based measurement of powder particles. “D90”, “D99”, and “D100” refer to cumulative 90% point of particle size (D90), cumulative 99% point of particle size (D99), and cumulative 100% point of particle size (D100) in volume based measurement of powder particles. Such a mean particle size is measured based on volume with a laser diffraction particle size distribution analyzer (e.g., Powrex Corp: PARTICLE VIEWER, Shimadzu Corp: SALD-3000J, or SYMPATEC: HELOS & RODOS). D100 can be derived from computation.

As used herein, “all . . . pass through . . . sieve” refers to either a case where 98% by weight or more of substance actually placed on a sieve pass through, or a case where the D99 particle size of each particle when measured by laser diffraction measurement is smaller than the mesh size of the sieve and is theoretically understood to pass through the sieve.

### (I) Component of Interest

The component of interest can be used without any particular limitation. Examples of “component of interest” used in the method of the present disclosure include active ingredients of medicaments or the like used in drugs, quasi-drugs, cosmetics, or the like, and components of agricultural chemicals, supplements, food products, or the like. A component of interest can also be used by mixing one or more components of interest. In specific embodiments in the food product industry, a product comprising the component of interest of the present disclosure can be used in a functional product, food for specified health uses, food with nutrient function claims, food with function claims, general food product, or the like.

A medicament can be used without any particular limitation. Any medicament or compound can be used as the “medicament” used in the method of the present disclosure, regardless of the property such as basic, acidic, amphoteric, or neutral, solubility, or heat resistance. Among them, it is preferable that a medicament is crystalline from the viewpoint of stability and ease of handling. A medicament can also be used by mixing one or more medicaments.

The component of interest used in the present disclosure can be any component of interest. Examples thereof include revitalizing health drug; antipyretic analgesic anti-inflammatory drug; antipsychotic drug; sedative hypnotic drug; antispasmodic; central nervous system agonist; cerebral metabolism improving drug; cerebral circulation improving drug; antiepileptic drug; sympathomimetic; digestant; antiulcer agent; gastrointestinal motility improving agent; antacid; antitussive expectorant; intestinal motility depressant; antiemetic agent; respiratory stimulant; bronchodilator; allergy drug; antihistamine; cardiotonic agent; arrhythmia agent; diuretic; ACE inhibitor; Ca antagonist; All antagonist; vasoconstrictor; coronary vasodilator; vasodilator; peripheral vasodilator; hyperlipidemia agent; cholagogue; cephem antibiotic; oral antimicrobial drug; chemotherapeutic agent; sulfonylurea drug; a glucosidase inhibitor; insulin sensitizer; fast-acting insulin secretagogue; DPP IV inhibitor; therapeutic agent for diabetic complications; osteoporosis agent; anti-rheumatic agent; skeletal muscle relaxant; alkaloid narcotic; sulfa agent; gout treating agent; blood coagulation inhibitor; antineoplastic agent; and the like.

Specific examples of the components of interest of the present disclosure include revitalizing health drugs such as vitamins, minerals, amino acids, crude drugs, and lactic acid bacteria; antipyretic analgesic anti-inflammatory drugs such as aspirin, acetaminophen, ethenzamide, ibuprofen, caffeine, and indomethacin; antipsychotic drugs such as blonanserin, lurasidone hydrochloride, tandospirone citrate, perospirone hydrochloride, reserpine, diazepam, fludiazepam, haloperidol, aripiprazole, and nortriptyline hydrochloride; sedative hypnotic drugs such as nitrazepam, diazepam, triazolam, brotizolam, zolpidem, and nimetazepam; antispasmodics such as scopolamine hydrobromide; central nervous system agonists such as zonisamide, droxidopa, citicoline, biperiden hydrochloride, and donepezil hydrochloride; cerebral metabolism improving drugs such as meclofenoxate hydrochloride; cerebral circulation improving drugs such as vinpocetine; antiepileptic drugs such as zonisamide, phenytoin, clonazepam, primidone, sodium valproate, carbamazepine, diazepam, ethotoin, and acetylpheneturide; sympathomimetics such as isoproterenol hydrochloride; digestants such as diastase, scopolia extract, and pancreatin; antiulcer agents such as cimetidine, lansoprazole, famotidine, sulpiride, and gefarnate; gastrointestinal motility improving agents such as mosapride citrate; antacids such as magnesium aluminometasilicate; antitussive expectorants such as cloperastine hydrochloride, ephedrine hydrochloride, and pentoxyverine citrate; intestinal motility depressants such as loperamide hydrochloride; antiemetic agents such as difenidol hydrochloride; respiratory stimulants such as levallorphan tartrate; bronchodilators such as theophylline; allergy drugs such as ebastine; antihistamines such as diphenhydramine hydrochloride; cardiotonic agents such as caffeine and digoxin; arrhythmia agents such as procainamide hydrochloride and arotinolol hydrochloride; diuretics such as isosorbide; ACE inhibitors such as delapril hydrochloride, captopril, and alacepril; Ca antagonists such as nifedipine, diltiazem hydrochloride, manidipine hydrochloride, and amlodipine besylate; All antagonists such as candesartan, olmesartan, and valsartan; vasoconstrictors such as phenylephrine hydrochloride; coronary vasodilators such as carbocromen hydrochloride; vasodilators such as limaprost alfadex; peripheral vasodilators such as cinnarizine; hyperlipidemia agents such as simvastatin and pravastatin sodium; cholagogues such as dehydrocholic acid; cephem antibiotics such as cephalexin and cefaclor; oral antimicrobial drugs such as gatifloxacin and sparfloxacin; chemotherapeutic agents such as sulfamethizole and pipemidic acid trihydrate; sulfonylurea drugs such as gliclazide, glibenclamide, and glimepiride; α glucosidase inhibitors such as acarbose, voglibose, and miglitol; insulin sensitizers such as pioglitazone hydrochloride and rosiglitazone; biguanide drugs such as metformin, buformin, and phenformin; fast-acting insulin secretagogues such as nateglinide and mitiglinide calcium hydrate; DPP IV inhibitors such as sitagliptin; therapeutic agents for diabetic complications such as ranirestat and epalrestat; osteoporosis agents such as etidronate disodium; anti-rheumatic agents such as methotrexate; skeletal muscle relaxants such as methocarbamol; antidizziness agents such as meclizine hydrochloride; alkaloid narcotics such as morphine hydrochloride and opium; sulfa agents such as sulfisomidine; gout treating agents such as allopurinol; blood coagulation inhibitors such as dicoumarol; antineoplastic agents such as 5-fluorouracil and mitomycin; and the like.

The component of interest in the present disclosure can be selected from indomethacin, blonanserin, lurasidone hydrochloride, tandospirone citrate, perospirone hydrochloride, fludiazepam, haloperidol, nortriptyline hydrochloride, nimetazepam, zonisamide, droxidopa, biperiden hydrochloride, phenytoin, clonazepam, primidone, sodium valproate, ethotoin, acetylpheneturide, pancreatin, cimetidine, sulpiride, gefarnate, mosapride citrate, ephedrine hydrochloride, pentoxyverine citrate, arotinolol hydrochloride, alacepril, amlodipine besylate, gatifloxacin, sparfloxacin, pipemidic acid trihydrate, gliclazide, miglitol, ranirestat, disodium etidronate, allopurinol, and the like.

When the present disclosure is used as a drug, the components of interest listed above can be in a salt or free form other than those described above, as long as they are pharmaceutically acceptable. The components of interest can also be in a form of a solvate such as an alcohol solvate or a hydrate. The blending ratio of a component of interest herein includes moisture of hydrate, solvent of solvate, and/or salt contained in the component of interest. The component of interest listed above can be used alone or as a combination of two or more. A component of interest which has been treated to mask an unpleasant taste such as bitterness can also be used. Examples of masking include coating of an active ingredient.

The mean particle size of components of interest is not particularly limited, and can change in the process of manufacturing component of interest-containing hollow particles or the like.

It is also possible to manufacture not only component of interest-containing hollow particles comprising a component of interest at a low content rate, but also those comprising a component of interest at a high content rate (e.g., 50 to 96% by weight, 55 to 70% by weight, 70 to 96% by weight, and 90 to 96% by weight per 100% by weight of the component of interest-containing hollow particle).

A component of interest can be in any part of a component of interest-containing hollow particle. Specifically, a component of interest can be in a nuclear particle, in a coating layer, between coating layers, or in the outermost layer.

(II) Macromolecule Contained in Nuclear Particle (Second Macromolecule)

A second macromolecule is defined in (II) Macromolecule contained in nuclear particle (second macromolecule), and a first macromolecule is defined in (VI) Macromolecule that is coatable microparticle (first macromolecule) herein, but these macromolecules can be the same or different macromolecules. When simply described as “macromolecule” herein, “macromolecule” can fall under both the first macromolecule and second macromolecule, as long as there is no inconsistency.

“A macromolecule” contained in a nuclear particle (second macromolecule) refers to a molecule with a large relative molecular mass, having a structure composed of numerous repeats of molecules with a small relative molecular mass, and refers especially to a functional macromolecule. The “molecule with a large relative molecular mass” refers to molecules with a mean molecular weight (weight average molecular weight: measured by light scattering method) of generally 1000 or greater, preferably 5000 or greater, and more preferably 10000 or greater. While the upper limit of molecular weight is not particularly limited, it is preferably 10000000 or less, more preferably 5000000 or less, still more preferably 2000000 or less, and especially preferably 1000000 or less. Examples of functional macromolecule include water soluble macromolecule, water insoluble macromolecule, enteric soluble macromolecule, and stomach soluble macromolecule. Preferred examples thereof include water soluble macromolecule, water insoluble macromolecule, enteric soluble macromolecule, and stomach soluble macromolecule. One or more second macromolecules can be mixed and used.

Examples of water insoluble macromolecule include water-insoluble cellulose ethers such as ethyl cellulose (e.g., trade name: Ethocel (Ethocel 10FP)) and cellulose acetate, water-insoluble acrylic acid copolymers such as aminoalkyl methacrylate copolymer RS (e.g., trade names: Eudragit RL 100, Eudragit RLPO, Eudragit RL 30 D, Eudragit RS 100, Eudragit RSPO, and Eudragit RS 30 D) and ethyl acrylate-methyl methacrylate copolymer dispersion (e.g., trade name: Eudragit NE 30 D), vinyl acetate resin, and the like. One or more can be mixed and used. Preferred examples thereof include ethyl cellulose and aminoalkyl methacrylate copolymer RS. The present disclosure can impart a function of sustained release or bitterness masking for a component of interest having bitterness by using a water insoluble macromolecule as the second macromolecule.

Examples of water soluble macromolecule include methyl cellulose (e.g., trade names: SM-4, SM-15, SM-25, SM-100, SM-400, SM-1500, SM-4000, 60SH-50, 60SH-4000, 60SH-10000, 65SH-50, 65SH-400, 65SH-4000, 90SH-100SR, 90SH-4000SR, 90SH-15000SR, and 90SH-100000SR), hydroxypropyl cellulose (e.g., trade names: HPC-SSL, HPC-SL, HPC-L, HPC-M, and HPC-H), hydroxypropyl methyl cellulose (e.g., trade names: TC5-E, TC5-M, TC5-R, TC5-S, and SB-4), hydroxyethyl cellulose (e.g., trade names: SP200, SP400, SP500, SP600, SP850, SP900, EP850, SE400, SE500, SE600, SE850, SE900, and EE820), hydroxylmethyl cellulose, and other cellulose derivatives and salts thereof, polyvinylpyrrolidone (e.g., trade names: Plasdone K12, Plasdone K17, Plasdone K25, Plasdone K29-32, Plasdone K90, and Plasdone K90D), polyvinyl alcohol (e.g., trade names: Gohsenol EG-05, Gohsenol EG-40, Gohsenol EG-05P, Gohsenol EG-05PW, Gohsenol EG-30P, Gohsenol EG-30PW, Gohsenol EG-40P, and Gohsenol EG-40PW), copolyvidone (e.g., trade names: Kollidon VA 64 and Plasdone S-630), polyethylene glycol, polyvinyl alcohol/acrylic acid/methyl methacrylate copolymer (e.g., trade name: POVACOAT), vinyl acetate/vinylpyrrolidone copolymer (e.g., trade name: Kollidon VA 64), polyvinyl alcohol/polyethylene glycol graft copolymer (e.g., trade name: Kollicoat IR), and other water soluble vinyl derivatives, pregelatinized starch (e.g., trade name: Amicol C), dextrin, dextran, pullulan, alginic acid, gelatin, pectin, and the like, one or more of which can be mixed and used. Preferred examples thereof include hydroxypropyl cellulose, hydroxypropyl methyl cellulose, polyvinylpyrrolidone, polyvinyl alcohol, and pregelatinized starch. More preferred examples thereof include hydroxypropyl cellulose. Use of a water soluble macromolecule as a second macromolecule in the present disclosure facilitates the achievement of complete medicament dissolution of reaching 100% medicament dissolution rate when a nuclear particle is applied with a sustained release coating using a water insoluble macromolecule as a first macromolecule.

Examples of enteric soluble macromolecule include hydroxypropyl methyl cellulose acetate succinate (e.g., trade names: AQOAT LF, AQOAT MF, AQOAT HF, AQOAT LG, AQOAT MG, and AQOAT HG), hydroxypropyl methyl cellulose phthalate (e.g., trade names: HPMCP 50, HPMCP 55, and HPMCP 55S), methacrylic acid copolymers such as methacrylic acid copolymer L (e.g., trade name: Eudragit L 100), methacrylic acid copolymer LD (e.g., trade name: Eudragit L 30D-55), dried methacrylic acid copolymer LD (e.g., trade name: Eudragit L 100-55), methacrylic acid copolymer S (e.g., trade name: Eudragit S 100), and methacrylic acid-N-butyl acrylate copolymer, and the like, one or more of which can be mixed and used. Preferred examples thereof include methacrylic acid copolymer L and dried methacrylic acid copolymer LD. In the present disclosure, dissolution of a component of interest within the stomach can be delayed by using an enteric soluble macromolecule as a second macromolecule.

Examples of stomach soluble macromolecule include stomach soluble polyvinyl derivatives such as polyvinyl acetal diethyl aminoacetate, stomach soluble acrylic acid copolymers such as aminoalkyl methacrylate copolymer E (e.g., trade names: Eudragit E 100 and Eudragit EPO) and the like, one or more of which can be mixed and used. Preferred examples thereof include aminoalkyl methacrylate copolymer E. In the present disclosure, bitterness due to dissolution of a component of interest in the mouth can be suppressed when an orally disintegrating tablet is designed by using a stomach soluble macromolecule as a second macromolecule.

In the present disclosure, a second macromolecule used as a raw material of a nuclear particle can be selected in accordance with the objective. To attain sustained release of a component of interest, it is preferable to use a water insoluble macromolecule as the second macromolecule. To achieve bitterness masking, it is preferable to use a water insoluble macromolecule, enteric soluble macromolecule, stomach soluble macromolecule, or the like. To suppress the dissolution of a component of interest in the stomach and to quicken the dissolution in the small intestine, it is preferable to use an enteric soluble macromolecule. An additional second macromolecule other than those described above can be used to form a complex, depending on the objective. For example, two or more second macromolecules with different functions such as a water soluble macromolecule and a water insoluble macromolecule can be mixed and used.

A second macromolecule in a particulate state is preferably used as a second macromolecule used in a nuclear particle. A second macromolecule with a suitable mean particle size or particle size distribution can be selected in accordance with the intended mean particle size or particle size distribution of component of interest-containing particles. A second macromolecule exemplified above includes those in a state of a dispersion, which can be used in the manufacture of a nuclear particle by, for example, spray drying or the like to prepare a particle and using the particle. To obtain, for example, component of interest-containing particles with a narrow particle size distribution, it is preferable to use second macromolecule with a narrow particle size distribution. To obtain component of interest-containing particles with a large mean particle size, it is preferable to use second macromolecule with a large mean particle size. To obtain component of interest-containing particles with a small mean particle size, it is preferable to use second macromolecule with a small mean particle size. Specifically, this means that component of interest-containing particles with a particle size distribution that matches the objective can be prepared by adjusting the size and particle size distribution of second macromolecule powder.

The amount of second macromolecule used as a raw material of a nuclear particle varies depending on the component of interest, amount of another additive, particle size, strength of binding force of the second macromolecule, or the like, but a second macromolecule is generally used in the range of 4 to 50% by weight, preferably 4 to 40% by weight, more preferably 6 to 40% by weight or 8 to 40% by weight, still more preferably 10 to 40% by weight, still yet more preferably 10 to 30% by weight, and especially preferably 10 to 20% by weight per 100% by weight of component of interest-containing hollow particles to be manufactured.

(III) Additives

The additives contained in a nuclear particle are not particularly limited, as long as they are additives that are commonly used. Examples thereof include excipients (e.g., starch such as rice starch, D-mannitol, and magnesium carbonate), binding agents, sweeteners, corrigents (taste or odor), flavoring agents, fluidizers (e.g., AEROSIL), antistatic agents, colorants, disintegrants, lubricants, plasticizers, deflocculating agents, coating agents, and the like. While the additive is not particularly limited, the additive can be blended without exerting a function of the second macromolecule of the present disclosure when the additive does not dissolve in the solvent used, even those falling under the second macromolecule described above.

(IV) Component of Interest-Containing Hollow Particle

Component of interest-containing hollow particles (see the “medicament-containing hollow particle” described in WO 2014/030656 as a representative example) refer to “particles consisting of a shell (or a wall) and a hollow section, comprising a component of interest and a macromolecule in the shell” or “particles having a structure with a hollow section surrounded by a wall consisting of a composition comprising a component of interest and a macromolecule”. If the component of interest is a medicament, the particle is referred to as a medicament-containing hollow particle. The particle can be referred to in the same manner for food ingredients and other components.

A component of interest and a macromolecule are essential constituents of a component of interest-containing hollow particle used as a nuclear particle. The particle refers to both a single particle and a collection of a plurality of particles.

The feature of component of interest-containing hollow particles is in having a hollow structure inside the particles. “Hollow” in such a case refers to a single completely independent vacancy at the center of a particle surrounded by a wall (shell) of a component of interest-containing composition, unlike a state of having numerous spaces without a defined position that is normally present in tablets. The presence thereof can be confirmed, for example, with an electron microscope or an optical microscope.

The ratio of the volume of a hollow section to the volume of the entire component of interest-containing hollow particle is preferably about 1% to 50%, more preferably 1% to 30%, still more preferably 1.5% to 30%, and most preferably about 2% to 30%. The volume ratio of a hollow section is found by dividing the volume of the hollow section by the volume of the particle. Since particles of the present disclosure generally have high spheroidicity, the volume is found by assuming that the hollow section and the particle are both spheres. The volumes of the hollow section and the particle can be computed by finding the major and minor axes of the particle and hollow section at the center of the particle by an X-ray CT (computerized tomographic device) and assuming the means thereof as the hollow section diameter and particle diameter to find the volume of the spheres.

More specifically, the “volume ratio of a hollow section” is found by calculation using the following equation.

Volume ratio of a hollow section [%]=(4/3×π×(diameter of hollow section/2)³)/(4/3×π×(particle size of component of interest-containing hollow particle/2)³)×100

The particle size of a component of interest-containing hollow particle and the diameter of a hollow section are non-destructively measured with a benchtop micro-CT scanner (SKYSCAN, SKYSCAN 1172). The mean value of 10 measurements is used.

Component of interest-containing hollow particles have a wall (shell) on the outside of a hollow section. The shell can have any thickness, but a thinner shell leads to weaker strength of the particle. The shell thickness of the present disclosure is preferably 10 μm or greater, more preferably 15 μm or greater, still more preferably 20 μm or greater, and most preferably 30 μm or greater. The shell thickness can be measured with, for example, an X-ray CT (computerized tomographic device).

The shell can have any percentage of thickness, which is found by the following equation. The percentage of shell thickness is preferably 20 to 80%, and more preferably 30 to 70%.

Percentage of shell thickness [%]=(shell thickness/(particle size of component of interest-containing hollow particle/2))×100

The feature of component of interest-containing hollow particles is in the ability to freely adjust the particle size. Therefore, particles can be adjusted to have a mean particle size of about 1 to 7000 μm, preferably about 5 to 1000 μm, more preferably about 10 to 500 μm, still more preferably about 10 to 400 μm, still more preferably about 20 to 300 μm, and most preferably about 50 to 300 μm.

From the viewpoint of particle strength, the particle size is preferably about 50 to 7000 μm, more preferably about 50 to 1000 μm, and still more preferably about 50 to 500 μm. From another viewpoint, particles can be adjusted to have a particle size of preferably about 70 to 7000 μm, more preferably about 70 to 1000 μm, still more preferably about 70 to 500 μm, especially preferably about 70 to 300 μm, and most preferably about 100 to 300 μm.

The size of component of interest-containing hollow particles can be adjusted by adjusting the mean particle size of second macromolecule.

The diameter of a hollow section is generally 10 μm or greater in a component of interest-containing hollow particle. The diameter of a hollow section can be adjusted freely, generally to about 10 to 5000 μm, preferably to about 20 to 700 μm, more preferably to about 30 to 300 μm, and still more preferably to about 50 to 200 μm. The ratio of the hollow section can be freely adjusted above in accordance with the particle size.

In one embodiment, a component of interest-containing hollow particle has a “smooth surface”. As used herein, smooth surface means absence of a protrusion, convexity, or concavity on the surface. Since fluidity of component of interest-containing hollow particles to be filled is required when filling the particles upon manufacturing tablets, capsules or the like, the component of interest-containing hollow particles preferably have a smooth surface. A component of interest-containing hollow particle preferably has a smooth surface because efficiency is enhanced when applying a coating to impart additional functionality to the component of interest-containing hollow particle. For example, such smoothness of surface can be observed visually. For visual observation, the particle can be magnified with a microscope or the like for observation. The evaluation thereof is expressed as “very smooth” (+++), “smooth” (++), “somewhat smooth” (+), and “not smooth” (−). “Very smooth” represents absence of a clear protrusion on the particle surface, or any convexity or concavity on the surface. “Smooth” represents absence of a clear protrusion on the particle surface, but the surface has gentle convexity or concavity. “Somewhat smooth” represents presence of a clear protrusion or clear convexity or concavity on the particle surface. “Not smooth” represents presence of a clear protrusion and clear convexity or concavity on the particle surface. The component of interest-containing hollow particle of the present disclosure may be “not smooth”, but is preferably “very smooth”, “smooth”, or “somewhat smooth”, more preferably “very smooth” or “smooth”, and still more preferably “very smooth”. 3D laser scanning confocal microscope VK-X200 (KEYENCE) can be used for the measurement. The “smooth surface” specifically means that the surface roughness (Ra value) measured by the tool described above is 3.5 or less, preferably 2.5 or less, and more preferably 1.5 or less.

The surface smoothness is affected by the ratio of mean particle sizes of second macromolecule and components of interest and/or another additive.

In one embodiment, a component of interest-containing hollow particle is spherical. As used herein, “spherical” refers to an aspect ratio of 1.0 to 1.5, preferably 1.0 to 1.4, and more preferably 1.0 to 1.3. Having such a shape, component of interest-containing hollow particles exhibit good fluidity when filled during the manufacture of a tablet, capsule, etc., and the efficiency is also improved during processing such as coating.

Component of interest-containing hollow particles are preferably those comprising 1 to 70% by weight of component of interest, 1 to 30% by weight of first macromolecule and second macromolecule, and 1 to 90% by weight of additive (including a lubricant) per 100% by weight of the component of interest-containing hollow particles.

The component of interest-containing hollow particles of the present disclosure are more preferably those comprising 5 to 50% by weight of component of interest, 1 to 40% by weight of first macromolecule and second macromolecule, and 5 to 80% by weight of additive (including a lubricant) per 100% by weight of the component of interest-containing hollow particles.

The component of interest-containing hollow particles of the present disclosure are still more preferably those comprising 10 to 40% by weight of component of interest, 10 to 40% by weight of first macromolecule and second macromolecule, and 10 to 70% by weight of additive (including a lubricant) per 100% by weight of the component of interest-containing hollow particles.

The component of interest-containing hollow particles of the present disclosure are most preferably those comprising 15 to 30% by weight of component of interest, 10 to 30% by weight of first macromolecule and second macromolecule, and 20 to 60% by weight of additive (including a lubricant) per 100% by weight of the component of interest-containing hollow particles.

The mean particle size of second macromolecule used as a raw material is generally 5-fold or greater, preferably 10-fold or greater, more preferably 15-fold or greater, still more preferably 20-fold or greater, and most preferably 25-fold or greater with respect to the mean particle size of components of interest and/or additive (including a lubricant) used as a raw material. The mean particle size is generally 1000-fold or less, preferably 500-fold or less, and more preferably 100-fold or less. Component of interest-containing hollow particles can be manufactured in accordance with the method described in WO 2014/030656 “medicament-containing hollow particle” to attain a given particle size.

Furthermore, it is preferable that the particle size distribution of second macromolecule used as a raw material does not overlap with the particle size distribution of components of interest and/or additive (including a lubricant) used as a raw material. Specifically, cumulative 10% point of particle size D10 in volume based measurement of second macromolecule is preferably greater than the cumulative 90% point of particle size D90 of component of interest and/or additive. In other words, cumulative 10% point of particle size D10 of second macromolecule is preferably 1-fold or greater, more preferably 2-fold or greater, and still more preferably 4-fold or greater with respect to the cumulative 90% point of particle size D90 of the component of interest and/or additive (including a lubricant). The cumulative 10% point of particle size D10 is also generally 5000000-fold or less.

Component of interest-containing hollow particles are preferably those comprising 1 to 70% by weight of component of interest and 1 to 30% by weight of macromolecule (more preferably those comprising 5 to 50% by weight of component of interest and 1 to 40% by weight of macromolecule, still more preferably those comprising 10 to 40% by weight of component of interest and 10 to 40% by weight of macromolecule; and most preferably those comprising 15 to 30% by weight of component of interest and 10 to 30% by weight of macromolecule) per 100% by weight of the component of interest-containing hollow particles, wherein a “preferred mean particle size of second macromolecule used as a raw material” is generally 10-fold or greater (preferably 15-fold or greater and more preferably 25-fold or greater) with respect to the mean particle size of the components of interest used as a raw material.

Component of interest-containing hollow particles are those comprising 1 to 70% by weight of component of interest, 1 to 30% by weight of macromolecule, and 1 to 90% by weight of additive for component of interest-containing hollow particles (more preferably those comprising 5 to 50% by weight of component of interest, 1 to 40% by weight of macromolecule, and 5 to 80% by weight of additive (including a lubricant), still more preferably those comprising 10 to 40% by weight of component of interest, 10 to 40% by weight of macromolecule, and 10 to 70% by weight of additive (including a lubricant), and most preferably those comprising 15 to 30% by weight of component of interest, 10 to 30% by weight of macromolecule, and 20 to 60% by weight of additive (including a lubricant)) per 100% by weight of the component of interest-containing hollow particles, wherein a preferred mean particle size of macromolecule used as a raw material is 10-fold or greater (preferably 15-fold or greater and more preferably 25-fold or greater) with respect to the mean particle size of powder mix of the component of interest and another additive used as a raw material.

(V) Nuclear Particle

As used herein, nuclear particle refers to all particles coated with macromolecule powder in the coating step of this technology. For example, when a component of interest-containing hollow particle obtained in the coating step of the present disclosure is used again in the coating step of the present disclosure, such a component of interest-containing hollow particle is also considered a nuclear particle in the new step.

Nuclear particles may or may not comprise a component of interest. Examples of component of interest include, but are not particularly limited to, medicaments, drugs, quasi-drugs, cosmetics, agricultural chemicals, supplements, and food products.

(VI) Macromolecule that is Coatable Microparticle (First Macromolecule)

As used herein, “microparticle” has a size equal to or less than “particle”. “Particle” and “microparticle” are used in the normal meaning of the art. In relation to the present disclosure, “particle” indicates especially those comprising a component of interest, and “microparticle” indicates those for coating. For this reason, the terms are used as in “particle coated with a coatable microparticle” herein. In such a case, the “particle” comprises a component of interest, a macromolecule, and the like in addition to “coatable microparticle”.

The first macromolecule in the present disclosure is preferably used as a solid, and is pulverized for use when the particle size is large. A first macromolecule can be pulverized alone, or co-pulverized with a small amount of dispersant. Examples of dispersants include low substituted hydroxypropyl cellulose, hydroxypropyl cellulose, hydroxypropyl methyl cellulose, hydroxypropyl cellulose acetate succinate, carboxymethyl cellulose, sodium carboxymethyl cellulose, carboxymethylcellulose calcium, crystalline cellulose, and other cellulose derivatives, polyvinylpyrrolidone/polyvinyl acetate, polyvinylpyrrolidone/polyvinyl alcohol, polyvinyl alcohol/PEG, polyvinyl caprolactam/polyvinyl acetate/polyethylene glycol, and other copolymers, colloidal silicon dioxide, silicon dioxide, magnesium aluminosilicate, microporous silica gel, polyorganosiloxane, medicinal clay, barium sulfate, talc, and other inorganic materials, crosslinked polyvinylpyrrolidone, crosslinked sodium carboxymethyl cellulose, β-cyclodextrin, α-cyclodextrin, hydroxypropyl-β-cyclodextrin, and other complexing agents, polyvinylpyrrolidone, hyaluronic acid, chitosan, xanthan, sodium alginate, polyvinyl acetate, sodium starch glycolate, lactose, sucrose fatty acid ester, and the like. A first macromolecule can be co-pulverized with a lubricant described below. First macromolecule exemplified below includes those in a state of a liquid dispersion, which can be used in the present disclosure by, for example, spray drying or the like to prepare a powder and then using the powder.

“First macromolecule” in the present disclosure can be any macromolecule that can adhere to the outer shell of a nuclear particle and laminate with a lubricant.

The mean molecular weight of first macromolecule (weight average molecular weight: measured by light scattering method) is generally 1000 or greater, preferably 5000 or greater, and more preferably 10000 or greater. The upper limit of molecular weight is not particularly limited, but is preferably 10000000 or less, more preferably 5000000 or less, still more preferably 2000000 or less, and especially preferably 1000000 or less.

In the step of coating component of interest-containing hollow particles, the mean particle size of nuclear particles is 5-fold or greater, preferably 10-fold or greater, more preferably 15-fold or greater, still more preferably 20-fold or greater, and especially preferably 25-fold or greater, and generally 10000000-fold or less with respect to the mean particle size of powdered first macromolecule. Since a macromolecule cannot be pulverized alone, a macromolecule comprises a dispersant, but the amount of dispersant is an amount that is substantially negligible with respect to the particle size of the macromolecule, so that the particle size of a macromolecule including a dispersant can be considered as the particle size of the macromolecule.

The D50 value of the powdered first macromolecule of the present disclosure is preferably less than 100 μm, less than 90 μm, less than 80 μm, less than 70 μm, less than 60 μm, less than 50 μm, less than 40 μm, less than 30 μm, less than 20 μm, or less than 10 μm. The D50 value of the powdered first macromolecule of the present disclosure is preferably 100 μm or less, 90 μm or less, 80 μm or less, 70 μm or less, 60 μm or less, 50 μm or less, 40 μm or less, 30 μm or less, 20 μm or less, or 10 μm or less. The D50 value of the powdered first macromolecule of the present disclosure is preferably 0.5 μm or greater, 0.8 μm or greater, 1 μm or greater, or 1.5 μm or greater. The D50 value of the powdered first macromolecule of the present disclosure is preferably greater than 0.5 μm, greater than 0.8 μm, greater than 1 μm, or greater than 1.5 μm.

The D90 value of the powdered first macromolecule of the present disclosure is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D90 value of the powdered first macromolecule of the present disclosure is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D90 value of the powdered first macromolecule of the present disclosure is preferably 1 μm or greater, 2 μm or greater, 3 μm or greater, or 4 μm or greater. The D90 value of the powdered first macromolecule of the present disclosure is preferably greater than 1 μm, greater than 2 μm, greater than 3 μm, or greater than 4 μm.

The D99 value of the powdered first macromolecule is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D99 value of the powdered first macromolecule of the present disclosure is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D99 value of the powdered first macromolecule of the present disclosure is preferably 1 μm or greater, 3 μm or greater, 5 μm or greater, or 7 μm or greater. The D99 value of the powdered first macromolecule of the present disclosure is preferably greater than 1 μm, greater than 3 μm, greater than 5 μm, or greater than 7 μm.

The D100 value of the powdered first macromolecule of the present disclosure is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D100 value of the powdered first macromolecule of the present disclosure is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D100 value of the powdered first macromolecule of the present disclosure is preferably 2 μm or greater, 5 μm or greater, 7 μm or greater, or 10 μm or greater. The D100 value of the powdered first macromolecule of the present disclosure is preferably greater than 2 μm, greater than 5 μm, greater than 7 μm, or greater than 10 μm.

The mean particle size of the powdered first macromolecule of the present disclosure is less than 50 μm, less than 45 μm, less than 40 μm, less than 35 μm, less than 30 μm, less than 25 μm, less than 20 μm, less than 15 μm, or less than 10 μm. The mean particle size of the powdered first macromolecule of the present disclosure is 50 μm or less, 45 μm or less, 40 μm or less, 35 μm or less, 30 μm or less, 25 μm or less, 20 μm or less, 15 μm or less, or 10 μm or less.

All of the powdered first macromolecule of the present disclosure can pass through a 100 mesh, 170 mesh, 200 mesh, 500 mesh, or 635 mesh sieve.

Examples of powdered first macromolecule include functional macromolecule. Examples of functional macromolecule include water soluble macromolecule, water insoluble macromolecule, enteric soluble macromolecule, and stomach soluble macromolecule. Preferred examples thereof include water soluble macromolecule, water insoluble macromolecule, enteric soluble macromolecule, and stomach soluble macromolecule. One or more first macromolecules can be mixed and used.

Examples of water soluble macromolecule include methyl cellulose (e.g., trade names: SM-4, SM-15, SM-25, SM-100, SM-400, SM-1500, SM-4000, 60SH-50, 60SH-4000, 60SH-10000, 65SH-50, 65SH-400, 65SH-4000, 90SH-100SR, 90SH-4000SR, 90SH-15000SR, and 90SH-100000SR), hydroxypropyl cellulose (e.g., trade names: HPC-SSL, HPC-SL, HPC-L, HPC-M, and HPC-H), hydroxypropyl methyl cellulose (e.g., trade names: TC5-E, TC5-M, TC5-R, TC5-S, and SB-4), hydroxyethyl cellulose (e.g., trade names: SP200, SP400, SP500, SP600, SP850, SP900, EP850, SE400, SE500, SE600, SE850, SE900, and EE820), hydroxylmethyl cellulose, and other cellulose derivatives and salts thereof, polyvinylpyrrolidone (e.g., trade names: Plasdone K12, Plasdone K17, Plasdone K25, Plasdone K29-32, Plasdone K90, and Plasdone K90D), polyvinyl alcohol (e.g., trade names: Gohsenol EG-05, Gohsenol EG-40, Gohsenol EG-05P, Gohsenol EG-05PW, Gohsenol EG-30P, Gohsenol EG-30PW, Gohsenol EG-40P, and Gohsenol EG-40PW), copolyvidone (e.g., trade names: Kollidon VA 64 and Plasdone S-630), polyethylene glycol, polyvinyl alcohol/acrylic acid/methyl methacrylate copolymer (e.g., trade name: POVACOAT), vinyl acetate/vinylpyrrolidone copolymer (e.g., trade name: Kollidon VA 64), polyvinyl alcohol/polyethylene glycol graft copolymer (e.g., trade name: Kollicoat IR), and other water soluble vinyl derivatives, pregelatinized starch (e.g., trade name: Amicol C), dextrin, dextran, pullulan, alginic acid, gelatin, pectin, and the like, one or more of which can be mixed and used. Preferred examples thereof include hydroxypropyl cellulose, hydroxypropyl methyl cellulose, polyvinylpyrrolidone, polyvinyl alcohol, and pregelatinized starch. More preferred examples include hydroxypropyl cellulose. Use of a water soluble macromolecule as a first macromolecule in the present disclosure enables impartation of a function of preventing particle destruction due to tableting pressure upon manufacture of a tablet containing the particle of the present disclosure, function of increasing hardness of a tablet, function of improving the taste of an orally disintegrating tablet, or a fast release function.

Examples of water insoluble first macromolecule include water-insoluble cellulose ethers such as ethyl cellulose (e.g., trade name: Ethocel (Ethocel 10P)) and cellulose acetate, water-insoluble acrylic acid copolymers such as aminoalkyl methacrylate copolymer RS (e.g., trade names: Eudragit RL 100, Eudragit RLPO, Eudragit RL 30 D, Eudragit RS 100, Eudragit RSPO, and Eudragit RS 30 D) and ethyl acrylate-methyl methacrylate copolymer dispersion (e.g., trade name: Eudragit NE 30 D), vinyl acetate resin, and the like, one or more of which can be mixed and used. Preferred examples thereof include ethyl cellulose and aminoalkyl methacrylate copolymer RS. The present disclosure can impart a function of sustained release or bitterness masking for a component of interest having bitterness by using a water insoluble macromolecule as the first macromolecule.

Examples of enteric soluble first macromolecule include hydroxypropyl methyl cellulose acetate succinate (e.g., trade names: AQOAT LF, AQOAT MF, AQOAT HF, AQOAT LG, AQOAT MG, and AQOAT HG), hydroxypropyl methyl cellulose phthalate (e.g., trade names: HPMCP 50, HPMCP 55, and HPMCP 55S), methacrylic acid copolymers such as methacrylic acid copolymer L (e.g., trade name: Eudragit L 100), methacrylic acid copolymer LD (e.g., trade name: Eudragit L 30D-55), dried methacrylic acid copolymer LD (e.g., trade name: Eudragit L 100-55), methacrylic acid copolymer S (e.g., trade name: Eudragit S 100), and methacrylic acid-N-butyl acrylate copolymer, and the like, one or more of which can be mixed and used. Preferred examples thereof include methacrylic acid copolymer L and dried methacrylic acid copolymer LD. In the present disclosure, dissolution of a component of interest within the stomach can be delayed by using an enteric soluble macromolecule as a first macromolecule.

Examples of stomach soluble first macromolecule include stomach soluble polyvinyl derivatives such as polyvinyl acetal diethyl aminoacetate, stomach soluble acrylic acid copolymers such as aminoalkyl methacrylate copolymer E (e.g., trade name: Eudragit E 100 and Eudragit EPO), and the like, one or more of which can be mixed and used. Preferred examples thereof include aminoalkyl methacrylate copolymer E. In the present disclosure, bitterness due to dissolution of a component of interest in the mouth can be suppressed when an orally disintegrating tablet is designed by using a stomach soluble macromolecule as a first macromolecule.

(VII) Coatable Lubricant

A coatable lubricant used in coating in the present disclosure can be any particle that can be laminated on the outer shell of a nuclear particle with a first macromolecule. A more preferred lubricant has a high bulk density. Specifically, the bulk density is preferably 0.1 g/mL or greater. The bulk density of a lubricant can be 0.2 g/mL or greater, 0.3 g/mL or greater, 0.4 g/mL or greater, or 0.5 g/mL or greater. A property of retaining homogeneity of mixture with a particle of a first macromolecule upon coating (particle size is not bulky) is preferred. The bulk density is measured using a graduated cylinder in accordance with the bulk density and tapped density testing method specified in the revised 16th Japanese Pharmacopoeia.

In the step of coating component of interest-containing hollow particles, the mean particle size of nuclear particles is 5-fold or greater, preferably 10-fold or greater, more preferably 15-fold or greater, still more preferably 20-fold or greater, and especially preferably 25-fold or greater, and generally 10000000-fold or less with respect to the mean particle size of a lubricant.

The D50 value of the lubricant of the present disclosure is preferably less than 100 μm, less than 90 μm, less than 80 μm, less than 70 μm, less than 60 μm, less than 50 μm, less than 40 μm, less than 30 μm, less than 20 μm, or less than 10 μm. The D50 value of the lubricant of the present disclosure is preferably 100 μm or less, 90 μm or less, 80 μm or less, 70 μm or less, 60 μm or less, 50 μm or less, 40 μm or less, 30 μm or less, 20 μm or less, or 10 μm or less. The D50 value of the coatable microparticle of the present disclosure is preferably 0.5 μm or greater, 0.8 μm or greater, 1 μm or greater, or 1.5 μm or greater. The D50 value of the coatable microparticle of the present disclosure is preferably greater than 0.5 μm, greater than 0.8 μm, greater than 1 μm, or greater than 1.5 μm.

The D90 value of the lubricant of the present disclosure is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D90 value of the lubricant of the present disclosure is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D90 value of the coatable microparticle of the present disclosure is preferably 1 μm or greater, 2 μm or greater, 3 μm or greater, or 4 μm or greater. The D90 value of the coatable microparticle of the present disclosure is preferably greater than 1 μm, greater than 2 μm, greater than 3 μm, or greater than 4 μm.

The D99 value of the lubricant of the present disclosure is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D99 value of the lubricant of the present disclosure is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D99 value of the coatable microparticle of the present disclosure is preferably 1 μm or greater, 3 μm or greater, 5 μm or greater, or 7 μm or greater. The D99 value of the coatable microparticle of the present disclosure is preferably greater than 1 μm, greater than 3 μm, greater than 5 μm, or greater than 7 μm.

The D100 value of the lubricant of the present disclosure is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D100 value of the lubricant of the present disclosure is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D100 value of the coatable microparticle of the present disclosure is preferably 2 μm or greater, 5 μm or greater, 7 μm or greater, or 10 μm or greater. The D100 value of the coatable microparticle of the present disclosure is preferably greater than 2 μm, greater than 5 μm, greater than 7 μm, or greater than 10 μm.

The mean particle size of the lubricant of the present disclosure is less than 50 μm, less than 45 μm, less than 40 μm, less than 35 μm, less than 30 μm, less than 25 μm, less than 20 μm, less than 15 μm, or less than 10 μm. The mean particle size of the lubricant of the present disclosure is 50 μm or less, 45 μm or less, 40 μm or less, 35 μm or less, 30 μm or less, 25 μm or less, 20 μm or less, 15 μm or less, or 10 μm or less.

All of the lubricant of the present disclosure can pass through a 100 mesh, 170 mesh, 200 mesh, 500 mesh, or 635 mesh sieve.

Examples of the lubricant include celluloses, lactose, lactose hydrate, saccharose, purified saccharose, purified licorice extract powder, glucose, D-mannitol, rice starch, corn starch, stearic acid, stearate, talc, oil and fat, metal oxide, fumaric acid, stearyl fumarate salt, alginic acid, alginate, ascorbic acid, aspartame, L-aspartic acid, xylitol, citric acid, citric acid hydrate, calcium citrate, sodium citrate, sodium citrate hydrate, glycine, D-xylose, L-glutamic acid, succinic acid, tartaric acid, sodium tartrate, sucralose, D-sorbitol, tannic acid, trehalose, peppermint powder, maltose hydrate, D-borneol, anhydrous citric acid, 1-menthol, DL-menthol, menthol powder, green tea powder, caramel, DL-malic acid, medicinal carbon, pigment, flavoring agent, benzoic acid, sodium benzoate, copper sulfate, calcium phosphate, calcium chloride, sodium phosphate, sodium chloride, calcium citrate, calcium carbonate, magnesium carbonate, calcium sulfate, magnesium chloride, sodium hydrogencarbonate, hydrous silicon dioxide, magnesium silicate, light anhydrous silicic acid, synthetic aluminum silicate, heavy anhydrous silicic acid, anhydrous silicic acid hydrate, anhydrous calcium phosphate, silicon dioxide, potassium sodium tartrate, sodium polyphosphate, metasilicic acid, aluminum sulfate, precipitated calcium carbonate, and zinc chloride. Specific examples of celluloses include crystalline cellulose, microcrystalline cellulose, crystalline cellulose carmellose sodium, carmellose, carmellose sodium, carmellose calcium, low substituted hydroxypropyl cellulose, and the like. Specific examples of stearate include sodium stearate, potassium stearate, zinc stearate, calcium stearate, aluminum stearate, magnesium stearate, polyoxyl stearate, and the like. Specific examples of oil and fat include hydrogenated castor oil, white petrolatum, polyoxyethylene powder, hydrogenated oil, cacao oil, hard wax, sodium lauryl sulfate, carnauba wax, oleic acid, rice starch, carrageenan, sucrose fatty acid ester, polyoxyethylene hydrogenated castor oil, beeswax, light fluidized paraffin, cetanol, and the like. Specific examples of metal oxides include iron oxides such as Yellow Ferric Oxide, Red Ferric Oxide, black iron oxide, brown iron oxide, and yellow iron oxide, titanium oxides, and the like. Specific examples of stearyl fumarate salt include sodium stearyl fumarate. Specific examples of alginate include sodium alginate.

Preferred examples thereof include magnesium aluminosilicate, celluloses, stearic acid, stearate, talc, metal oxide, stearyl fumarate salt, talc, Red Ferric Oxide, Yellow Ferric Oxide, titanium oxide, sodium stearyl fumarate, sodium stearate, hydrogenated oil, magnesium stearate, and crystalline cellulose. Still more preferred examples thereof include magnesium aluminosilicate, talc, Red Ferric Oxide, Yellow Ferric Oxide, titanium oxide, sodium stearyl fumarate, and magnesium stearate.

The lubricant in the present disclosure is pulverized for use when the particle size is large. A lubricant can be pulverized alone, or co-pulverized with a powdered first macromolecule.

In one embodiment, the weight ratio of a first macromolecule to a lubricant is between 1:10 and 10:1, preferably 1:5 and 5:1. The weight ratio of a first macromolecule to a lubricant can be 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1, or a value between any combination of these weight ratios.

In one embodiment, the D50 value of a particle produced with a first macromolecule and an additive (before adding a lubricant) is preferably less than 100 μm, less than 90 μm, less than 80 μm, less than 70 μm, less than 60 μm, less than 50 μm, less than 40 μm, less than 30 μm, less than 20 μm, or less than 10 μm. The D50 value of a particle produced with a first macromolecule and an additive (before adding a lubricant) is preferably 100 μm or less, 90 μm or less, 80 μm or less, 70 μm or less, 60 μm or less, 50 μm or less, 40 μm or less, 30 μm or less, 20 μm or less, or 10 μm or less. The D50 value of a particle produced with a first macromolecule and an additive (before adding a lubricant) is preferably 0.5 μm or greater, 0.8 μm or greater, 1 μm or greater, or 1.5 μm or greater. The D50 value of a particle produced with a first macromolecule and an additive (before adding a lubricant) is preferably greater than 0.5 μm, greater than 0.8 μm, greater than 1 μm, or greater than 1.5 μm.

The D90 value of a particle produced with a first macromolecule and an additive (before adding a lubricant) is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D90 value of a particle produced with a first macromolecule and an additive (before adding a lubricant) is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D90 value of a particle produced with a first macromolecule and an additive (before adding a lubricant) is preferably 1 μm or greater, 2 μm or greater, 3 μm or greater, or 4 μm or greater. The D90 value of a particle produced with a first macromolecule and an additive (before adding a lubricant) is preferably greater than 1 μm, greater than 2 μm, greater than 3 μm, or greater than 4 μm.

In one embodiment, the D50 value of a particle produced with a first macromolecule and a lubricant is preferably less than 100 μm, less than 90 μm, less than 80 μm, less than 70 μm, less than 60 μm, less than 50 μm, less than 40 μm, less than 30 μm, less than 20 μm, or less than 10 μm. The D50 value of a particle produced with a first macromolecule and a lubricant is preferably 100 μm or less, 90 μm or less, 80 μm or less, 70 μm or less, 60 μm or less, 50 μm or less, 40 μm or less, 30 μm or less, 20 μm or less, or 10 μm or less. The D50 value of a particle produced with a first macromolecule and a lubricant is preferably 0.5 μm or greater, 0.8 μm or greater, 1 μm or greater, or 1.5 μm or greater. The D50 value of a particle produced with a first macromolecule and a lubricant is preferably greater than 0.5 μm, greater than 0.8 μm, greater than 1 μm, or greater than 1.5 μm.

The D90 value of a particle produced with a first macromolecule and a lubricant is preferably less than 200 μm, less than 190 μm, less than 180 μm, less than 170 μm, less than 160 μm, less than 150 μm, less than 140 μm, less than 130 μm, less than 120 μm, less than 110 μm, less than 100 μm, less than 90 μm, less than 80 μm, less than 70 μm, less than 60 μm, less than 50 μm, less than 40 μm, less than 30 μm, less than 20 μm, or less than 10 μm. The D90 value of a particle produced with a first macromolecule and a lubricant is preferably 200 μm or less, 190 μm or less, 180 μm or less, 170 μm or less, 160 μm or less, 150 μm or less, 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, or 10 μm or less. The D90 value of a particle produced with a first macromolecule and a lubricant is preferably 1 μm or greater, 2 μm or greater, 3 μm or greater, or 4 μm or greater. The D90 value of a particle produced with a first macromolecule and a lubricant is preferably greater than 1 μm, greater than 2 μm, greater than 3 μm, or greater than 4 μm.

(VIII) Component of Interest-Containing Hollow Particle of the Present Disclosure

The component of interest-containing hollow particles of the present disclosure are those comprising 0.1 to 95.9% by weight of component of interest, 4 to 40% by weight of second macromolecule used as a raw material of a nuclear particle, 0.1 to 95.9% by weight of powdered first macromolecule, and 0.1 to 95.9% by weight of a lubricant; preferably those comprising 1 to 94% by weight of component of interest, 5 to 30% by weight of second macromolecule used as a raw material of a nuclear particle, 1 to 94% by weight of additive, 1 to 94% by weight of powdered first macromolecule, and 1 to 94% by weight of a lubricant; or those comprising 10 to 80% by weight of component of interest, 10 to 20% by weight of second macromolecule used as a raw material of a nuclear particle, 10 to 80% by weight of additive, 10 to 80% by weight of powdered first macromolecule, and 10 to 80% by weight of a lubricant, per 100% by weight of the component of interest-containing hollow particles.

Examples of the component of interest-containing hollow particles of the present disclosure include those comprising 60 to 96% by weight of component of interest and to 40% by weight of second macromolecule (preferably those comprising 70 to 95% by weight of component of interest and 5 to 30% by weight of second macromolecule, more preferably those comprising 80 to 90% by weight of component of interest and 10 to 20% by weight of second macromolecule) per 100% by weight of the component of interest-containing hollow particles, wherein a preferred mean particle size of a powdered first macromolecule and a lubricant is 5-fold or greater (preferably 15-fold or greater and more preferably 25-fold or greater) with respect to the mean particle size of coatable microparticle.

Examples of the component of interest-containing hollow particles of the present disclosure include those comprising 55 to 95.9% by weight of component of interest, 4 to 40% by weight of second macromolecule, and 0.1 to 5% by weight of additive (preferably those comprising 65 to 94.9% by weight of component of interest, 5 to 30% by weight of second macromolecule, and 0.1 to 5% by weight of additive, more preferably those comprising 75 to 89.9% by weight of component of interest and 10 to 20% by weight of second macromolecule) per 100% by weight of the component of interest-containing hollow particles, wherein a preferred mean particle size of nuclear particles is 5-fold or greater (preferably 15-fold or greater and more preferably 25-fold or greater) with respect to the mean particle size of a powdered first macromolecule and a lubricant.

Another embodiment thereof includes those comprising 75 to 89.9% by weight of component of interest and 10 to 20% by weight of second macromolecule, wherein a preferred mean particle size of nuclear particles is 2-fold or greater (preferably 5-fold or greater and more preferably 10-fold or greater) with respect to the D90 value of powdered first macromolecule and a lubricant.

A still another embodiment thereof includes those comprising 75 to 89.9% by weight of component of interest and 10 to 20% by weight of second macromolecule, wherein a preferred mean particle size of nuclear particles is 2-fold or greater (preferably 5-fold or greater and more preferably 10-fold or greater) with respect to the D100 value of a powdered first macromolecule and a lubricant.

A still another embodiment thereof includes those comprising 75 to 89.9% by weight of component of interest and 10 to 20% by weight of second macromolecule, wherein a preferred mean particle size of nuclear particles is 2-fold or greater (preferably 5-fold or greater and more preferably 10-fold or greater) with respect to the D99 value of a powdered first macromolecule and a lubricant.

Examples of the component of interest-containing hollow particles of the present disclosure include those comprising 0.1 to 95.9% by weight of component of interest, to 40% by weight of second macromolecule, and 0.1 to 95.9% by weight of additive (preferably those comprising 1 to 94% by weight of component of interest, 5 to 30% by weight of second macromolecule, and 1 to 94% by weight of additive, more preferably those comprising 10 to 80% by weight of component of interest, 10 to 20% by weight of second macromolecule, and 10 to 80% by weight of additive) per 100% by weight of the component of interest-containing hollow particles, wherein a preferred mean particle size of nuclear particles is 5-fold or greater (preferably 15-fold or greater and more preferably 25-fold or greater) with respect to the mean particle size of a powdered first macromolecule and a lubricant.

Another embodiment thereof includes those comprising 10 to 80% by weight of component of interest, 10 to 20% by weight of second macromolecule, and 10 to 80% by weight of additive, wherein a preferred mean particle size of nuclear particles is 2-fold or greater (preferably 5-fold or greater and more preferably 10-fold or greater) with respect to the D90 value of a powdered first macromolecule and a lubricant.

A still another embodiment thereof includes those comprising 10 to 80% by weight of component of interest, 10 to 20% by weight of second macromolecule, and 10 to 80% by weight of additive, wherein a preferred mean particle size of nuclear particles is 2-fold or greater (preferably 5-fold or greater and more preferably 10-fold or greater) with respect to the D100 value of a powdered first macromolecule and a lubricant.

A still another embodiment thereof includes those comprising 10 to 80% by weight of component of interest, 10 to 20% by weight of second macromolecule, and 10 to 80% by weight of additive, wherein a preferred mean particle size of nuclear particles is 2-fold or greater (preferably 5-fold or greater and more preferably 10-fold or greater) with respect to the D99 value of a powdered first macromolecule and a lubricant.

The component of interest-containing hollow particles of the present disclosure can be high performance component of interest-containing hollow particles. For example, fast-release, enteric soluble, stomach soluble, sustained release, or bitterness masking function or the like is improved.

In one embodiment, a first macromolecule and a lubricant can be coated, for example, at 10% by weight to 50% by weight, 10% by weight to 60% by weight, 10% by weight to 70% by weight, 10% by weight to 80% by weight, 10% by weight to 90% by weight, or 10% by weight to 100% by weight, or at 100% by weight or more, with respect to a nuclear particle of the component of interest-containing hollow particle of the present disclosure. The ratio of a first macromolecule and a lubricant to nuclear particle can be 10% by weight, 11% by weight, 12% by weight, 13% by weight, 14% by weight, 15% by weight, 16% by weight, 17% by weight, 18% by weight, 19% by weight, 20% by weight, 21% by weight, 22% by weight, 23% by weight, 24% by weight, 25% by weight, 26% by weight, 27% by weight, 28% by weight, 29% by weight, 30% by weight, 31% by weight, 32% by weight, 33% by weight, 34% by weight, 35% by weight, 36% by weight, 37% by weight, 38% by weight, 39% by weight, 40% by weight, 41% by weight, 42% by weight, 43% by weight, 44% by weight, 45% by weight, 46% by weight, 47% by weight, 48% by weight, 49% by weight, 50% by weight, 55% by weight, 60% by weight, 65% by weight, 70% by weight, 75% by weight, 80% by weight, 85% by weight, 90% by weight, 95% by weight, 100% by weight, 105% by weight, 110% by weight, 115% by weight, 120% by weight, 125% by weight, 130% by weight, 135% by weight, 140% by weight, 145% by weight, or 150% by weight, of a value between any combination of these values.

The present disclosure provides a composition for imparting a function of a macromolecule to a component of interest-containing hollow particle consisting of a shell and a hollow section, comprising the macromolecule and a lubricant. The component of interest-containing hollow particle can comprise a second macromolecule and a component of interest, and the composition can comprise a first macromolecule and a lubricant. The present disclosure also provides a composition comprising a lubricant for imparting a function of a first macromolecule to a component of interest-containing hollow particle consisting of a shell and a hollow section, wherein the component of interest-containing hollow particle comprises a second macromolecule and a component of interest, and the first macromolecule is provided with the lubricant. The function comprises fast release, sustained release, enteric solubility, stomach solubility, bitterness masking, or photostability.

The first macromolecule and the lubricant of the present disclosure can enhance the property of a second macromolecule contained in an inner core. A particle coated with the first macromolecule and the lubricant of the present disclosure can improve, for example, fast release property, enteric solubility, stomach solubility, sustained release property, and bitterness masking. When the first macromolecule and the lubricant of the present disclosure are used, high performance coated component of interest-containing hollow particles can be made efficiently and in a short period of time.

Manufacturing Method

The manufacturing method of a particle coated with a powdered first macromolecule and a lubricant of the present disclosure comprises the steps of (1) preparing a nuclear particle comprising a component of interest and a second macromolecule, and (2) adding the first macromolecule and the lubricant to the nuclear particle, and coating the mixture while spraying a solvent that can dissolve the first macromolecule. The manufacturing method of a particle coated with a first macromolecule and a lubricant of the present disclosure is a method that is simple yet has excellent coatability (coating time and coverage (release controlling ability)).

The step of (1) preparing a nuclear particle comprising a component of interest and a second macromolecule of the present disclosure obtains a nuclear particle in a wet powder state by loading a “second macromolecule” and “component of interest” into a granulator as powder and granulating while spraying a predetermined amount of solvent under specific mixing/granulating conditions. In the present disclosure, a nuclear particle can be used in the next step while still in a wet powder state or used after drying by fluidized bed drying or the like.

The step of (2) adding the first macromolecule and the lubricant to the nuclear particle and coating the resulting mixture by spraying a solvent that can dissolve the first macromolecule while rolling the mixture of the present disclosure can be performed by adding the first macromolecule and the lubricant to the nuclear particle in a wet powder state or dry state described above and coating the mixture while spraying a predetermined amount of solvent that can dissolve the first macromolecule under a specific coating condition, which would roll the mixture. The resulting particles in a wet powder state can be dried by fluidized bed drying or the like.

A coating method can be appropriately selected from granulation methods having a function for rolling nuclear particles during coating. For example, particles can be manufactured using a stirring granulation method, mixing stirring granulation method, high-speed stirring granulation method, high-speed mixing stirring granulation method, rolling and stirring fluidized bed granulation method, or rolling granulation method. In particular, it is preferable to use a stirring granulation method, mixing stirring granulation method, high-speed stirring granulation method, or high-speed mixing stirring granulation method. Examples of granulators (including container rotating granulators) that are used for stirring granulation, mixing stirring granulation, or the like include Intensive Mixer (Nippon Eirich), versatile mixer (Shinagawa Machinery Works), Super mixer (Kawata Mfg. Co., Ltd.), FM mixer (Nippon Coke & Engineering Co., Ltd.), SPG series (Fuji Paudal Co, Ltd.), Vertical Granulator (e.g., models FM-VG-05 and FM-VG-100, Powrex Corp), High-speed agitating mixer and granulator Pharma Matrix (Nara Machinery Co., Ltd.), high-speed mixer (FUKAE POWTEC Co, Ltd.), Granumeist (Freund Corporation), New-Gra Machine (Seishin Enterprise Co., Ltd.), Triple Master (Shinagawa Machinery Works), and the like. In the present disclosure, a simple fluidized bed granulation method is not preferable because the drying efficiency is too high such that granulation would not progress.

As a drying method, a known method can be appropriately selected. Examples thereof include drying using a rack dryer or fluidized bed and the like. Drying using a fluidized bed is preferable from the viewpoint of manufacturability.

If the particle size of powdered first macromolecule is greater than a desired size, the macromolecule is pulverized for use. A pulverizer is not particularly limited, as long as it is capable of pulverizing first macromolecule. Examples thereof include roller pulverizers such as roller mills and edge liners, tumbler mills such as ball mills and tower mills, high speed impact mills such as pin mills and hammer mills, and fluid energy mills such as jet mills. While powdered first macromolecule can be pulverized alone, powdered first macromolecule can be mixed with a small amount of a dispersant and co-pulverized. Powdered first macromolecule can also be mixed with a lubricant and co-pulverized.

If the particle size of a lubricant is greater than a desired size, the lubricant is pulverized for use. A pulverizer is not particularly limited, as long as it is capable of pulverizing a lubricant. Examples thereof include roller pulverizers such as roller mills and edge liners, tumbler mills such as ball mills and tower mills, high speed impact mills such as pin mills and hammer mills, and fluid energy mills such as jet mills. While a lubricant can be pulverized alone, a lubricant can be mixed with powdered first macromolecule and co-pulverized.

Any mixing method can be appropriately selected, as long as the method has a mixing function. For example, a diffusion mixer such as a tumbler mixer, V blender, or W blender, or a convection mixer such as a ribbon mixer, Nauta mixer, or planetary mixer can be used.

Any tableting method of the component of interest-containing hollow particle of the present disclosure can be appropriately selected, as long as the method has a function of compression molding a powder. Examples thereof include a tableting apparatus classified as a tablet press. A lubricant can also be added to the tablet of the present disclosure by an external lubrication method.

“Solvent” in the present disclosure refers to all acceptable solvents in the art for a drug, quasi-drug, cosmetic, food product, or the like. Solvent can be any solvent that can dissolve a second macromolecule or first macromolecule to be used. A pharmaceutically acceptable solvent is preferred from the viewpoint of using the component of interest-containing hollow particle of the present disclosure as a drug. Such a solvent can be appropriately selected in accordance with the types of component of interest, macromolecule, or additive or the like. Several types of solvent can be mixed and used.

Examples of “solvent” in the present disclosure include water, alcohol based solvents (e.g., methanol, ethanol, n-propyl alcohol, isopropyl alcohol, 2-methoxyethanol, 2-ethoxyethanol, and other optionally substituted lower alkanol), ketone based solvents (e.g., acetone, methyl ethyl ketone, and other lower alkyl ketone), ester based solvents (e.g., ethyl acetate ester and other lower alkyl esters of acetic acid) and mixtures thereof.

Specifically, the present disclosure can use a solvent that can dissolve a macromolecule (e.g., water, hydroalcoholic solvent, or the like) as the solvent when using a water soluble macromolecule as the macromolecule. Water or hydrous ethanol can be especially preferably used. The present disclosure can also use a solvent that can dissolve a macromolecule (e.g., alcohol based solvent, ketone based solvent, ester based solvent, or the like) as the solvent when using a water insoluble macromolecule as the macromolecule. The present disclosure can use a solvent that can dissolve each of the macromolecule including enteric soluble macromolecule, stomach soluble macromolecule, and chitosan (e.g., alcohol based solvent, more specifically ethanol) as the solvent.

While the amount of solvent used upon coating of the present disclosure varies by the type or amount of component of interest or macromolecule or the like, the amount is generally 5 to 60% by weight, preferably 10 to 53% by weight, more preferably 10 to 40% by weight, and still more preferably 15 to 40% by weight per 100% by weight of the total amount of each component constituting a particle. The solvent is preferably added to a powder mixture comprising a nuclear particle, a powdered first macromolecule, and a lubricant by spraying.

A solvent can be sprayed, upon coating of the present disclosure, using a spray gun that is generally used for granulation. Specific examples thereof include a needle spray gun (Tomita engineering Co., Ltd.) and the like. To enhance the granulation yield, it is preferable to spray a solvent as little as possible to parts other than the powder within a granulation container, i.e., to the inner wall of the granulation container or the like, and to spray the solvent in as broad of a range of powder within the granulation container as possible.

While the amount of solvent used in the manufacture of a nuclear particle varies by the type or amount of component of interest or macromolecule or the like, the amount is generally 5 to 60% by weight, preferably 10 to 53% by weight, more preferably 10 to 40% by weight, and still more preferably 15 to 40% by weight per 100% by weight of the total amount of each component constituting a particle. The solvent is preferably added to a powder mixture comprising a component of interest and macromolecule by spraying.

A solvent can be sprayed in the manufacture of a nuclear particle by using a spray gun that is generally used for granulation. Specific examples thereof include a needle spray gun (Tomita engineering Co., Ltd.) and the like. To enhance the granulation yield, it is preferable to spray a solvent as little as possible to parts other than the powder within a granulation container, i.e., to the inner wall of the granulation container or the like, and to spray the solvent in as broad of a range of powder within the granulation container as possible. Further, the mist size of a sprayed solvent is preferably narrow because the solvent disperses more uniformly onto powder with a smaller mist size. Meanwhile, if the spray pressure is increased in order to reduce the mist size, powder would scatter to inhibit the rolling motion. Thus, it is preferable to reduce the mist size of a solvent with a suitable mist pressure setting.

When using an additive in the manufacture of a nuclear particle, the mean particle size of powder mixture of a component of interest and/or additive used as a raw material is important for the manufacture of coated component of interest-containing hollow particles. In such a case, the mean particle size of second macromolecule used as a raw material is 5-fold or greater, preferably 10-fold or greater, more preferably 15-fold or greater, and especially preferably 25-fold or greater with respect to the mean particle size of powder mixture of a component of interest and/or additive used as a raw material. The mean particle size is also generally 1000-fold or less, preferably 500-fold or less, and more preferably 100-fold or less.

Furthermore, the particle size distribution of second macromolecule used as a raw material preferably does not overlap with the particle size distribution of a powder mixture of a component of interest and/or additive used as a raw material. Specifically, cumulative 10% point of particle size D10 in volume base measurement of second macromolecule used as a raw material is preferably, for example, greater than the cumulative 90% point of particle size D90 of a powder mixture of a component of interest and/or additive used as a raw material. In other words, cumulative 10% point of particle size D10 of second macromolecule used as a raw material is preferably 1-fold or greater (i.e., ratio of particle size distributions of second macromolecule to component of interest and/or additive (D10/D90) is 1-fold or greater), more preferably 2-fold or greater, and still more preferably 4-fold or greater with respect to the cumulative 90% point of particle size D90 of a powder mixture of the component of interest and additive used as a raw material. The cumulative 10% point of particle size D10 is also generally 500-fold or less, preferably 250-fold or less, and more preferably 50-fold or less.

For example, the cumulative 50% point of particle size D50 in volume base measurement of second macromolecule used as a raw material is preferably greater than the cumulative 50% point of particle size D50 of a powder mixture of a component of interest and/or additive used as a raw material. In other words, the cumulative 50% point of particle size D50 of second macromolecule used as a raw material is preferably 1-fold or greater (i.e., ratio of particle size distributions of second macromolecule to component of interest (D50/D50) is 1-fold or greater), more preferably 2-fold or greater, and still more preferably 4-fold or greater with respect to the cumulative 50% point of particle size D50 of a powder mixture of the component of interest and/or additive used as a raw material. The cumulative 50% point of particle size D50 is also generally 500-fold or less, preferably 250-fold or less, and more preferably 50-fold or less.

Characteristic Values

The “aspect ratio” in the present disclosure is a ratio of the minor diameter to the major diameter of a particle, and is an indication of the sphericity. The aspect ratio can be determined by calculation using, for example, the following formula.

Aspect ratio=major diameter of particle/minor diameter of particle

The major diameter and minor diameter of a particle are non-destructively measured with a benchtop micro-CT scanner (SKYSCAN, SKYSCAN 1172), and the mean value of 10 measurements is used.

In addition, Millitrac JPA (NIKKISO CO., LTD.) can be used for the measurement.

The “particle size distribution width” in the present disclosure can be found from the ratio of cumulative 90% point of particle size D90 to cumulative 10% point of particle size D10 (D90/D10) in volume based measurement of powdered particles. The particle size distribution of the component of interest-containing hollow particles of the present disclosure can be conveniently adjusted by adjusting the particle size of second macromolecule. For example, a particle group having a narrow particle size distribution width can be produced. Such particle size distribution width is measured with a laser diffraction particle size analyzer (POWREX CORPORATION, Particle Viewer) in the basis of volume.

In the present disclosure, “particle size distribution width is narrow” means that a specific particle size distribution width (D90/D10) is 6.0 or less, preferably 5.0 or less, more preferably 4.0 or less, and still more preferably 3.0 or less.

The strength of a hollow particle can be evaluated by particle shell strength. The “particle shell strength” in the present disclosure can be found by calculation using the following equation.

Particle shell strength [MPa]=2.8P/(π×d²−π×d′²)×1000

P: particle destruction testing force [mN], d: diameter of component of interest-containing hollow particle [μm], d′: diameter of hollow section [μm]

Such a particle destruction testing force and diameter of component of interest-containing hollow particle are measured with SHIMADZU Micro Compression Testing Machine MCT-W500 (Shimadzu Corporation).

A “diameter of a hollow section” in the present disclosure can be found by calculation using the following equation.

Diameter of hollow section [μm]=(major diameter of hollow section+minor diameter of hollow section)/2

The major diameter and minor diameter of the hollow section of the particle are non-destructively measured with a benchtop micro-CT scanner (SKYSCAN, SKYSCAN 1172), and the mean value of 10 measurements is used.

In the present disclosure, a component of interest-containing hollow particle desirably has a sufficient particle strength to be efficiently coated without being broken or chipped, even when it is coated with a functional macromolecule and the like to impart an additional function by using a fluidized-bed granulator, various microparticle coating machines, or the like that require further mechanical strength of particles, and maintain a hollow structure without being crushed even after tableting.

The component of interest-containing hollow particles of the present disclosure have sufficient particle strength. Since the component of interest-containing hollow particles have a hollow section, a conventional particle strength measurement method cannot perform an accurate evaluation due to calculation of the hollow section as a solid. Thus, the particle shell strength excluding the hollow section can be measured. The “sufficient particle strength” in the present disclosure specifically means that the particle shell strength of a component of interest-containing particle is 2.0 MPa or less, preferably 3.0 MPa or less, more preferably 4.0 MPa or less, and still more preferably 5.0 MPa or less.

“Particle size of component of interest-containing hollow particle” in the present disclosure can be found by calculation using the following equation.

The particle size of a component of interest-containing hollow particle can be found by calculation using the following equation.

Particle size of component of interest-containing hollow particle [μm]=(major diameter of particle+minor diameter of particle)/2

The major diameter and minor diameter of the particle are non-destructively measured with a benchtop micro-CT scanner (SKYSCAN, SKYSCAN 1172), and the mean value of 10 measurements is used.

The “shell thickness” in the present disclosure can be found by calculation using the following equation.

Shell thickness [μm]=(particle size of component of interest-containing hollow particle−diameter of hollow section)/2

The particle size of a component of interest-containing hollow particle and the diameter of a hollow section are non-destructively measured with a benchtop micro-CT scanner (SKYSCAN, SKYSCAN 1172), and the mean value of 10 measurements is used.

The “percentage of shell thickness” in the present disclosure can be found by calculation using the following equation.

Percentage of shell thickness [%]=(shell thickness/(particle size of component of interest-containing hollow particle/2))×100

The particle size of a component of interest-containing hollow particle is non-destructively measured with a benchtop micro-CT scanner (SKYSCAN, SKYSCAN 1172), and the mean value of 10 measurements is used.

The “volume ratio of a hollow section” in the present disclosure can be found by calculation using the following equation.

Volume ratio of a hollow section [%]=(4/3×π×(diameter of hollow section/2)³)/(4/3×π×(particle size of component of interest-containing hollow particle/2)³)×100

The particle size of a component of interest-containing hollow particle and the diameter of a hollow section are non-destructively measured with a benchtop micro-CT scanner (SKYSCAN, SKYSCAN 1172), and the mean value of 10 measurements is used.

The “ratio of particle size distributions of a second macromolecule to a component of interest (D50/D50)” in the present disclosure can be found by calculation using the following equation.

Ratio of particle size distributions of a second macromolecule to a component of interest (D50/D50)=D50 of a second macromolecule/D50 of a component of interest

The “ratio of particle size distributions of a second macromolecule to powder mixture of components of interest and other additives (D50/D50)” in the present disclosure can be found by calculation using the following equation.

Ratio of particle size distributions of a second macromolecule to powder mixture of components of interest and other additives (D50/D50)=D50 of second macromolecule/D50 of powder mixture of components of interest and other additives

The particle size distribution of a second macromolecule, components of interest, and powder mixture of components of interest and other additives is measured with a laser diffraction particle size analyzer (POWREX CORPORATION, Particle Viewer) or a laser diffraction particle size analyzer (Shimadzu Corporation, SALD-3000) or SYMPATEC, HELOS & RODOS) based on volume.

The “ratio of particle size distributions of a second macromolecule to a component of interest (D10/D90)” in the present disclosure can be found by calculation using the following equation.

Ratio of particle size distributions of a second macromolecule to a component of interest (D10/D90)=D10 of second macromolecule/D90 of components of interest

The “ratio of particle size distributions of a second macromolecule to powder mixture of components of interest and other additives (D10/D90)” in the present disclosure can be found by calculation using the following equation.

Ratio of particle size distributions of a second macromolecule to powder mixture of components of interest and other additives (D10/D90)=D10 of second macromolecule/D90 of powder mixture of components of interest and other additives

The particle size distribution of a second macromolecule, a component of interest, and powder mixture of components of interest and other additives is measured with a laser diffraction particle size analyzer (POWREX CORPORATION, Particle Viewer) or a laser diffraction particle size analyzer (Shimadzu Corporation, SALD-3000) or SYMPATEC, HELOS & RODOS) based on volume.

While a conventional method using a fluidized bed granulator requires several days or more as the coating time, the coating time is 1 hour or less when using the manufacturing method of the present disclosure. Since coating can be applied in a short period of time, production efficiency is enhanced.

The function of a first macromolecule can be added to the component of interest-containing hollow particles of the present disclosure, in addition to a function of a nuclear particle. For example, a particle with stomach insolubility in addition to the function of a second macromolecule contained in a nuclear particle can be manufactured by controlling the amount of coating using enteric soluble powdered first macromolecule. If a macromolecule with a sustained release property is used for first macromolecule, a component of interest-containing hollow particle having any sustained release profile (any 50% dissolution time) can be manufactured by controlling the amount of coating. Similarly, by using a macromolecule having stomach soluble or bitterness masking property in a nuclear particle, these functions can be controlled in any manner.

Degradation of a component of interest contained in a nuclear particle due to light can be suppressed by selecting microparticle with a photostable function as the lubricant. Examples of microparticle with a photostable function include titanium oxide, Red Ferric Oxide, Yellow Ferric Oxide, black iron oxide, pigment, and the like.

Pharmaceutical Composition and Application Thereof

The present disclosure relates to a pharmaceutical composition, therapeutic agent, and/or prophylactic agent for treating and/or preventing a digestive system disease or digestive system symptom, comprising the component of interest-containing hollow particle of the present disclosure. In an exemplary embodiment, the digestive system disease is a constipation-predominant irritable bowel syndrome (IBS) or chronic constipation. Examples of diseases that can be treated and/or prevented in the present disclosure include malignant lymphoma, atopic dermatitis, Alzheimer's disease, allergic rhinitis, gastric cancer, gastroesophageal reflux, addiction, hereditary arrhythmia, pharyngeal cancer, influenza, viral hepatitis, depression, ALS (amyotrophic lateral sclerosis), ulcerative colitis, overactive bladder, stiff shoulders, irritable bowel syndrome, hypersensitivity pneumonitis, pollinosis, age-related macular degeneration, age-related hearing loss, Kawasaki disease, hepatoma, liver cancer, interstitial pneumonia, rheumatoid arthritis, hallux valgus, ptosis, eye strain, functional dyspepsia, acute myeloid leukemia, acute nephropathy, acute pancreatitis, thoracic outlet syndrome, angina, anorexia, myopia, tension headache, subarachnoid hemorrhage, cluster headache, tuberculosis, vascular dementia, tenosynovitis, cuff tear, dysmenorrhea, premenstrual syndrome, premenstrual dysphoric disorder (PMDD), hypertension, eosinophilic sinusitis, halitosis, higher order brain dysfunction, laryngeal cancer, mouth ulcer, postmenopausal syndrome, depression among the elderly, osteonecrosis, osteomyelitis, osteoporosis, pelvic organ prolapse, pediatric depression, aspiration pneumonia, frozen shoulder, sarcopenia, tooth erosion, Sjögren's syndrome, uterine myoma, endometrial cancer, endometriosis, dyslipidemia, periodontal disease, fatty liver, carpal tunnel syndrome, small intestine cancer, food poisoning, esophageal cancer, food allergy, myocardial infarction, myocardial disease, heart failure, COPD (chronic obstructive pulmonary disease), hemorrhoids, juvenile dementia, kidney cancer, renal failure, hives, normal pressure hydrocephalus, spinal stenosis, scoliosis, dysphagia, fibromyalgia, systemic lupus erythematosus, asthma, vestibular neuronitis, frontotemporal dementia, prostate cancer, prostatic hypertrophy, bipolar disorder, shingles, multiple myeloma, cholelithiasis, gall bladder, bile duct cancer, colon cancer, aortic dissection, aortic aneurysm, central sleep apnea, herniated disk, gout, epilepsy, schizophrenia, diabetes, diabetic neuropathy, diabetic nephropathy, diabetic retinopathy, sudden sensorineural hearing loss, arteriosclerosis, dry mouth, NASH, narcolepsy, sarcoma, breast cancer, urinary stones, dementia, heat stroke, cerebral infarction, cerebral hemorrhage, brain tumor, stroke, norovirus, pneumonia, lung cancer, MAC lung disease, cataract, developmental disorder, syphilis, flexor tendonitis, Parkinson's disease, nonodontogenic toothache, skin cancer, anemia, rubella, paranasal sinusitis, arrhythmia, insomnia, obstructive sleep apnea, arteriosclerosis obliterans, herpesvirus, ankle osteoarthritis, shoulder osteoarthritis, osteoarthritis, hip osteoarthritis, knee osteoarthritis, migraine, tonsillitis, incontinence, constipation, cystitis, bladder cancer, Mycoplasma pneumoniae, ingrown nails, chronic purulent sinusitis, chronic myeloid leukemia, chronic kidney disease (CKD), chronic pancreatitis, chronic low back pain, taste disorder, unruptured cerebral aneurysm, tooth decay, asymptomatic cerebral infarction, restless legs syndrome, metabolic syndrome, Meniere's disease, moyamoya disease, lower back pain, mumps, benign paroxysmal positional vertigo, glaucoma, Lewy body dementia, and locomotive syndrome.

In the present disclosure, “prevention (prophylaxis)” is an act of administering the component of interest of the present disclosure, which is the active ingredient, to a healthy individual who has not developed a disease or is not in an unhealthy condition as of the administration. “Prophylactic agent” is administered to such a healthy individual. For example, a prophylactic agent is intended to prevent the development of a disease and is expected to be suitable for especially individuals who have had a symptom of a disease previously or individuals considered to be at increased risk of suffering from the disease. “Therapy” is an act of administering the component of interest of the present disclosure, which is an active ingredient, to an individual (patient) diagnosed to have developed a disease by a physician. “Therapeutic agent” is administered to such a patient. For example, a therapeutic agent is intended to alleviate a disease or symptom, prevent exacerbation of a disease or symptom, or restore the condition to that prior to developing the disease. Even when the objective of administration is prevention of exacerbation of a disease or symptom, this is an act of therapy if the agent is administered to a patient.

In the present disclosure, specific examples of “digestive system disease or digestive system symptom” include the diseases or symptoms of the following (i) to (iii).

(i) digestive system diseases such as irritable bowel syndrome, atonic constipation, habitual constipation, chronic constipation, constipation induced by agents such as morphine and antipsychotics, constipation accompanying Parkinson's disease, constipation accompanying multiple sclerosis, constipation accompanying diabetes, and constipation or defecation disorder due to a contrast agent (as pretreatment for an endoscopic examination or barium enteric enema X-ray examination);
(ii) digestive system diseases such as functional dyspepsia, acute/chronic gastritis, reflux esophagitis, gastric ulcer, duodenal ulcer, gastric neurosis, postoperative paralytic ileus, senile ileus, non-diffuse gastroesophageal reflux, NSAID ulcer, diabetic gastroparesis, post-gastrectomy syndrome, and intestinal pseudo-obstruction; and
(iii) digestive system symptoms such as anorexia, nausea, vomiting, bloating, epigastric discomfort, abdominal pain, heartburn, and eructation in the digestive system diseases described in (i) and (ii), scleroderma, diabetes, or esophagus/biliary tract disease.

The dosage form of the component of interest of the present disclosure can be either oral administration or parenteral administration. The dosage varies by the dosing method, patient's symptom, age, or the like, but is generally in the range of 0.01 to 30 mg/kg/day, preferably 0.05 to 10 mg/kg/day, and more preferably 0.1 to 3 mg/kg/day. Another preferred embodiment of the dosage is generally in a range of 0.01 mg to 1000 mg/day, preferably 0.1 mg to 500 mg/day, more preferably 0.5 mg to 300 mg/day, still more preferably 1 mg to 200 mg/day, and most preferably 5 mg to 100 mg/day. The number of daily doses is one or several per day, such as 1, 2, or 3 doses given each time.

Examples of the dosage form of an oral formulation include granules, tablets, capsules, suspension (aqueous suspension, oil suspension), emulsion, and the like. Examples of parenteral formulations include injection, intravenous drip agent, suppository (intrarectally administered agent), intranasal agent, sublingual agent, transdermally absorbed agent [lotion, emulsion, ointment, cream, jelly, gel, patch (tape, transdermal patch formulation, poultice, and the like), externally applied powder, and the like], and the like.

Preferably, the component of interest of the present disclosure is orally administered as the component of interest-containing hollow particle or formulation of the present disclosure. More preferable examples of the dosage form of oral formulation include tablets, as described in the formulation comprising a component of interest-containing hollow particle of present disclosure. More preferred examples of tablets include orally disintegrating tablets.

This includes combined therapy that administers the present compound or pharmaceutically acceptable salt thereof, or hydrate or solvate thereof, or component of interest-containing hollow particle, formulation, or pharmaceutical composition of the present disclosure in combination with one or more of the following additional agents, sequentially or simultaneously, for the treatment of a disease described herein.

For digestive system diseases accompanying constipation, specific examples include saline laxatives such as magnesium sulfate, magnesium oxide, and magnesium citrate, invasive laxatives such as dioctyl sodium, sulfosuccinate, and casanthranol, bulk-forming laxatives such as carmellose, intestine irritating laxatives such as bisacodyl, picosulfate, senna, and sennoside, small intestine irritating laxatives such as castor oil, bowel cleansing agents such as Magcorol and Niflec, and the like.

For digestive system diseases such as functional dyspepsia, acute/chronic gastritis, reflux esophagitis, non-diffuse gastroesophageal reflux, diabetic gastroparesis, gastric ulcer, duodenal ulcer, NSAID ulcer, gastric neurosis, postoperative paralytic ileus, senile ileus, post-gastrectomy syndrome, and intestinal pseudo-obstruction, examples thereof include proton pump inhibitors such as omeprazole, rabeprazole, and lansoprazole, antacids such as histamine H₂ receptor inhibitors such as famotidine, ranitidine, and cimetidine, gastrointestinal function regulators such as Mosapride and domperidone, gastric mucosa protective agents, intestinal regulators, and the like.

### EXAMPLES

The present disclosure is specifically described in more detail with Examples, Test Examples, and Comparative Examples, but the present disclosure is not limited thereto. The present disclosure can also be modified to the extent that the modified invention remains within the scope of the present disclosure. The compound names denoted in the following Examples, Test Examples, and Comparative Examples do not necessarily follow the IUPAC nomenclature.

Unless specifically noted otherwise, % in solvent indicates (W/W %) and % in particles indicate % by weight in the Examples, Test Examples, and Comparative Examples.

The following components were used in the Examples and Comparative Examples, unless specifically noted otherwise.

Aminoalkyl methacrylate copolymer RS (Eudragit RSPO): Evonik Degussa Japan Co., Ltd.

Dried methacrylic acid copolymer LD (Eudragit L100-55): Evonik Degussa Japan Co., Ltd.

Talc (Micro Ace® P-3): Nippon Talc, Co., Ltd.

Titanium oxide (Titanium oxide (NA61): Toho Titanium Co., Ltd.

Sodium stearyl fumarate (PRUV®: Rettenmaier Japan Co., Ltd.

Magnesium aluminosilicate (Neusilin UFL2): Fuji Chemical Industries Co., Ltd.

Aminoalkyl methacrylate copolymer E (Eudragit E 100): Evonik Degussa Japan Co., Ltd.

Ethylcellulose (Ethocel 10FP): Dow Chemical Japan Limited

Hydroxypropyl cellulose (HPC-L): Nippon Soda Co., Ltd.

Magnesium aluminometasilicate (Neusilin UFL2): Fuji Chemical Industries Co., Ltd.

<Testing Method>

The testing methods in the Examples, Test Examples, and Comparative Examples are the following.

(Particle Size Distribution)

The particle size distribution of coating mixtures comprising a first macromolecule and a lubricant was measured with a laser diffraction particle size distribution analyzer (SYMPATEC: HELOS & RODOS) based on volume. The D50 and D90 values were extracted from measurement data.

The particle size distribution of a component of interest, a macromolecule (including a first macromolecule and a second macromolecule), other additives, powder mixture of components of interest and other additives, and resulting component of interest-containing hollow particles was measured with a laser diffraction particle size distribution analyzer (e.g., SYMPATEC: HELOS & RODOS) based on volume. The D50, D90, D99, and D100 values were extracted or computed from measurement data.

(Appearance of Component of Interest-Containing Hollow Particle)

The appearance of particles was observed with a scanning electron microscope (Hitachi, Ltd., model S-3400N).

(50% Dissolution Time)

50% dissolution time was computed from the following equation.

50% dissolution time=(maximum dissolution test sample point where dissolution rate does not exceed 50%)+((50−(dissolution rate at maximum dissolution test sample point where dissolution rate does not exceed 50%))/((dissolution rate at minimum dissolution test sample point where dissolution rate exceeds 50%)−(dissolution rate at maximum dissolution test sample point where dissolution rate does not exceed 50%))/((minimum dissolution test sample point where dissolution rate exceeds 50%)−(maximum dissolution test sample point where dissolution rate does not exceed 50%))

<Active Pharmaceutical Ingredient>

The following were used as the active pharmaceutical ingredients in the Examples, Test Examples, and Comparative Examples, unless specifically noted otherwise.

Zonisamide (1,2-BENZISOXAZOLE-3-METHANESULFONAMIDE; hereinafter compound A)

Acetaminophen (N-(4-Hydroxyphenyl)acetamide; hereinafter compound B)

Anhydrous caffeine (1,3,7-Trimethyl-1H-purine-2,6(3H,7H)-dione; hereinafter compound C)

### Example 1<Manufacture of Component of Interest-Containing Hollow Particles with Different Amounts of Coating>

The component of interest-containing hollow particles of the present disclosure with different amounts of coating were manufactured in Examples 1-1 and 1-2. As shown in Table 1, the amount of coating was selected as 20% by weight or 40% by weight with respect to a nuclear particle to be coated. First, a mixture of a representative example of a first macromolecule, dried methacrylic acid copolymer LD, and magnesium aluminosilicate (mass ratio: dried methacrylic acid copolymer LD:magnesium aluminosilicate=1:0.05) was pulverized with a spiral jet mill (100 AS, Hosokawa Micron Corporation) to obtain particle mixture for coating 1. The mean particle size (D50) of the mixture at this time was about 14.7 μm, and the D90 was about 39.4 μm. 133.4 g of the particle mixture for coating 1 and 66.6 g of talc were then mixed to obtain mixture for coating 2. A second macromolecule, i.e., aminoalkyl methacrylate copolymer RSPO, were passed through a No. 100 sieve, and the residual on the sieve was used as aminoalkyl methacrylate copolymer RS (No. 100 on).

Nuclear particles to be coated were manufactured in accordance with Table 1. Specifically, aminoalkyl methacrylate copolymer RS (representative example of a second macromolecule denoted as aminoalkyl methacrylate copolymer RS (No. 100 on) in Table 1) and compound A were loaded into a high-speed stirring granulator, i.e., Vertical Granulator (model FM-VG-05, capacity: 5L, Powrex Corp) at the amounts described in Table 1 as powder. Granulation was then performed while spraying the aqueous 95% ethanol solution (for nuclear particle) described in Table 1 under the mixing/granulation conditions shown in Table 2 to obtain nuclear particles to be coated in a wet powder state. The nuclear particles to be coated in a wet powder state were loaded into a fluidized bed dryer (MP-01, Powrex Corp) and dried under the drying conditions shown in Table 2 to obtain the nuclear particles to be coated. The nuclear particles to be coated were loaded into a high-speed stirring granulator, i.e., Vertical Granulator (model FM-VG-01, Powrex Corp), and coated while spraying the aqueous 95% ethanol solution (for coating) described in Table 1 under the mixing/coating conditions specified in Table 2 as particle mixture for coating 2 was separated and added 8 times at 25 g each. When particle mixture for coating 2 was added to an amount equivalent to 20% by weight (when 100 g of particle mixture for coating was added and coated), samples were collected. The sampled component of interest-containing hollow particles in a wet powder state were loaded into a rack dryer (Perfect Oven, ESPEC Corp.) and dried overnight at 50° C. to obtain the component of interest-containing hollow particles of Example 1-1. Subsequent to the sampling, the coating step was continued until 200 g of mixture for coating 3 was coated to obtain component of interest-containing hollow particles in a wet powder state. The component of interest-containing hollow particles in a wet powder state were loaded into a rack dryer (Perfect Oven, ESPEC Corp.) and dried overnight at 50° C. to obtain the component of interest-containing hollow particles of Example 1-2.

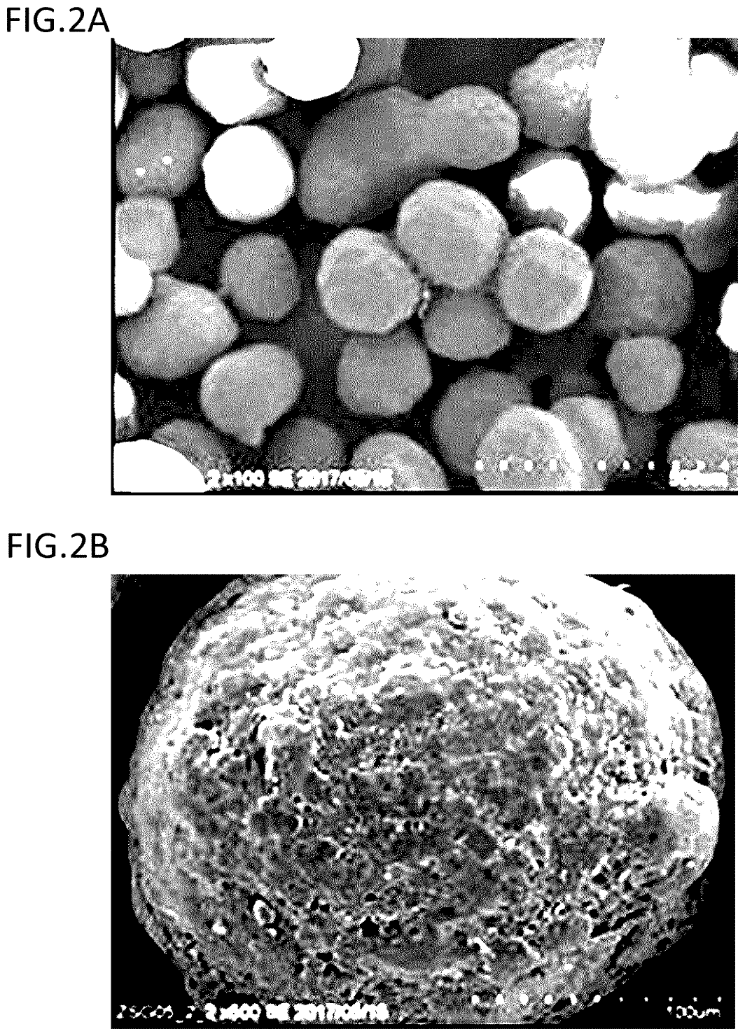

The coating time, and the time required for manufacture of the resulting particles are shown in Table 6. The appearances of the particles obtained in Example 1-1 are shown in FIGS. 2A and 2B.

TABLE 1

| | Comparative Example 1 | | Example 1-1 | | Example 1-2 | |
|---|---|---|---|---|---|---|
| | Amount | Formulation | Amount | Formulation | Amount | Formulation |
| | loaded | ratio | loaded | ratio | loaded | ratio |
| | (g) | (%) | (g) | (%) | (g) | (%) |
| Compound A | 560 | 80 | 400 | 66.7 | 400 | 57.1 |
| (ground product | | | | | | |
| of JM) | | | | | | |
| Aminoalkyl | 140 | 20 | 100 | 16.7 | 100 | 14.3 |
| methacrylate | | | | | | |
| copolymer RS | | | | | | |
| (No. 100 on) | | | | | | |
| Particle mixture | — | — | 66.7 | 11.1 | 133.4 | 19.1 |
| for coating 1 | | | | | | |
| (dried | | | | | | |
| methacrylic acid | | | | | | |
| copolymer | | | | | | |
| LD:magnesium | | | | | | |
| aluminosilicate = | | | | | | |
| 1:0.05) | | | | | | |
| Talc | | | 33.3 | 5.6 | 66.6 | 9.5 |
| (Aqueous 95% | (150) | (25) | — | — | — | — |
| ethanol solution) | | | | | | |
| (for nuclear | | | | | | |
| particle) | | | | | | |
| (Aqueous 95% | | — | (55) | (9.2) | (110) | (15.8) |
| ethanol solution) | | | | | | |
| (for coating) | | | | | | |
| Total | 700 | 100 | 600 | 100 | 700 | 100 |

TABLE 2

| | Equipment | Mixing | No. of blade | No. of chopper | |
|---|---|---|---|---|---|
| | name | time | rotations | rotations | |
| Premixing step | Vertical Granulator | 3 min | 400 min⁻¹ | 3000 min⁻¹ | |
| | (FM-VG-05, Powrex | | | | |
| | Corp) | | | | |
| | Equipment | No. of blade | No. of chopper | Solvent | Solvent |
| | name | rotations | rotations | addition method | addition rate |
| Granulation step | Vertical Granulator | 400 min⁻¹ | 3000 min⁻¹ | Spray addition | 8 g/min |
| | (FM-VG-05, Powrex | | | | |
| | Corp)) | | | | |
| | Equipment | No. of blade | No. of chopper | Solvent addition | Solvent |
| | name | rotations | rotations | method | addition rate |
| Coating step | Vertical Granulator | 450 min⁻¹ | 3000 min⁻¹ | Spray addition | 4 g/min |
| | (FM-VG-01, Powrex | | | | |
| | Corp) | | | | |

| | | Temperature | | Exhaust |
|---|---|---|---|---|
| | Equipment | of supplied | Amount of air | temperature after |
| | name | air | supplied | completion of drying |
| Drying step | Multiplex (MP-01, | 80° C. | 0.5 m³/min | 40° C. |
| | Powrex Corp) | | | |
| | Equipment name | Air temperature | Drying time | |
| | Rack dryer (Perfect | 50 | 12 hours | |
| | Oven, ESTEC Corp.) | | | |

### Comparative Example 1 Manufacture of Nuclear Particles to be Coated

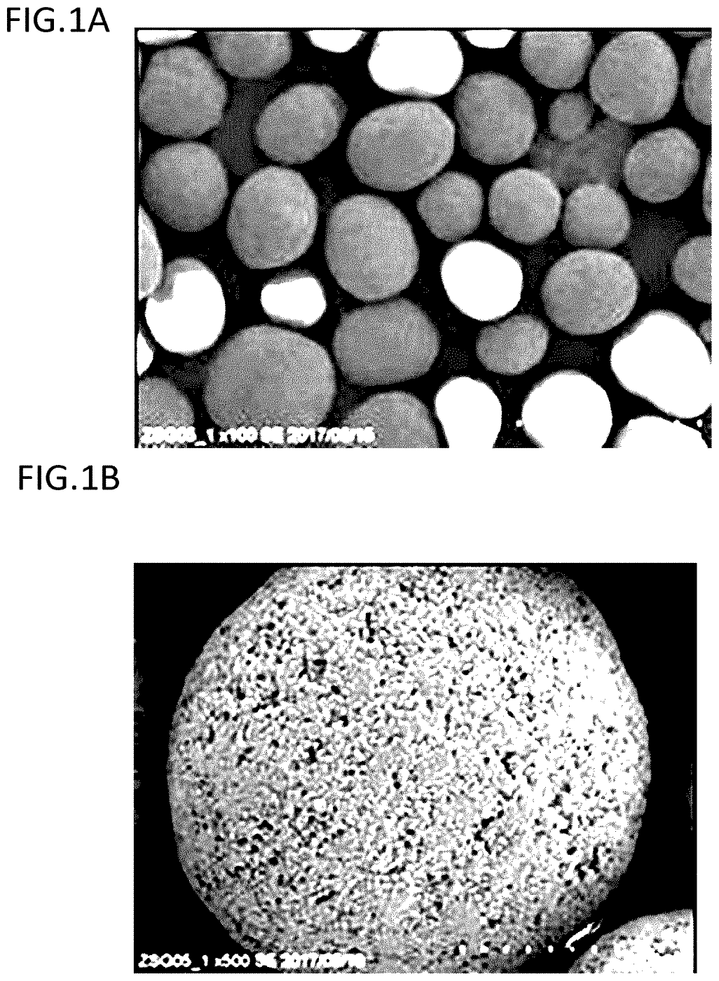

In Comparative Example 1, only particles that were not coated, i.e., nuclear particles to be coated, were manufactured in accordance with the formulation ratio and amount loaded described in Table 1 in the same manner as Example 1. After granulating nuclear particles to be coated in a wet powder state as in Example 1, the nuclear particles to be coated in a wet powder state were subjected to fluidized bed drying using a Multiplex (model MP-01, Powrex Corp) to obtain the nuclear particles to be coated of Comparative Example 1. The appearances of the resulting particles are shown in FIGS. 1A and 1B.

### Test Example 1<Dissolution Test on Tablets Comprising Component of Interest-Containing Hollow Particles with Different Amounts of Coating>

Dissolution tests were conducted using the particles manufactured in Comparative Example 1 and Examples 1-1 and 1-2. The amount of sample in the test was an amount equivalent to 100 components of interest. 37° C./900 ML of 1st fluid and 2nd fluid for dissolution test in the Japanese Pharmacopoeia was used as the test solution for measurement at 50 RPM based on the paddle method of a dissolution test method in the revised 16th Japanese Pharmacopoeia. The measurement times were 10, 15, 30, 45, 60, 90, 120, and 360 minutes. The sampling solution was filtered and measured by HPLC to compute the dissolution rate.

<HPLC Measurement Conditions>

Detector: ultraviolet visible spectrophotometer
Measurement wavelength: 285 NM

### Column: WATERS ACQUITY UPLC C18 [2.1 MM Φ×30 MM]

Column temperature: 40° C.
Flow rate: 0.5 ML/MIN
Injection volume: 5 ML
Sample cooler: 25° C.
Syringe cleansing solution: water/acetonitrile mixture=1/1
Mobile phase: water/acetonitrile mixture=4/1

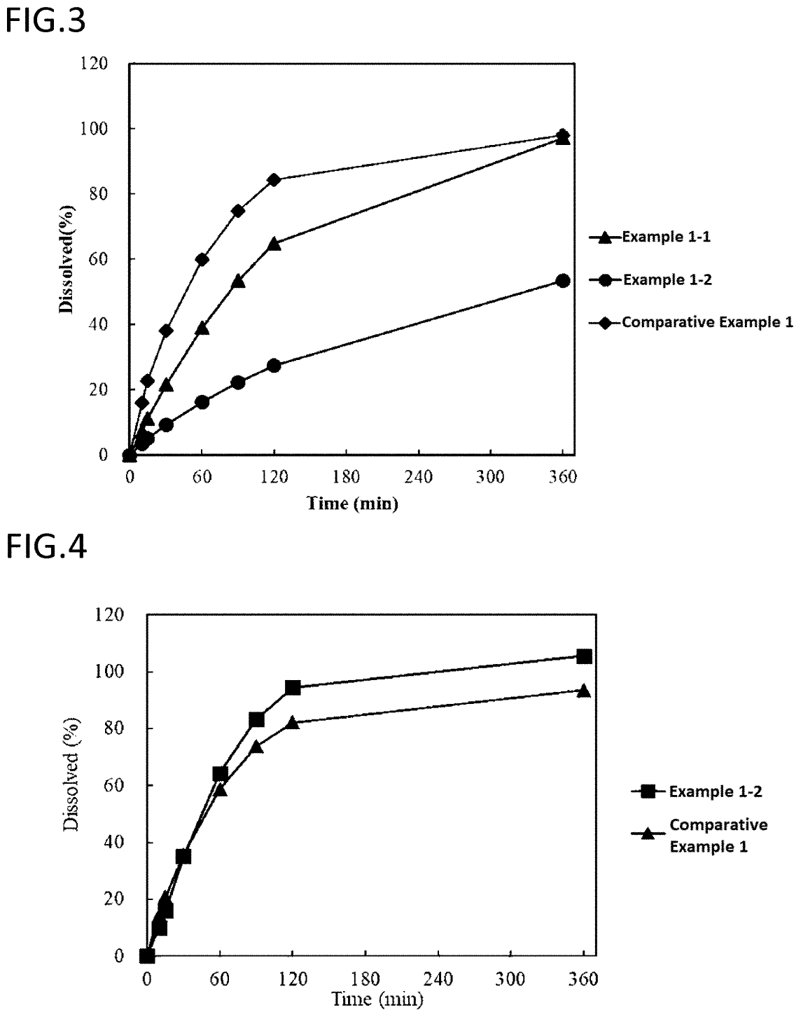

FIGS. 3 and 4 show the results of dissolution tests on the particles obtained in Comparative Example 1 and Examples 1-1 and 1-2, and Table 6 shows the ratio of 50% dissolution times before and after coating. FIG. 3 is the test result using 1st fluid for dissolution test, and FIG. 4 is the test result using 2nd fluid for dissolution test. The ability to control release of particles increased with the increase in the amount of coating.

### Example 2<Manufacture of Component of Interest-Containing Hollow Particles Using a Coatable First Microparticle and a Lubricant with Different Particle Sizes>

The component of interest-containing hollow particles of the present disclosure with different coatable microparticle sizes were manufactured in Example 2.

A dried methacrylic acid copolymer LD/talc mixture was used as a coatable first macromolecule and a deflocculating agent (lubricant) with different particle sizes. While D50 of the dried methacrylic acid copolymer LD/talc mixture in Example 1 was 6.5 μm and D90 was 24.1 μm, D50 of the dried methacrylic acid copolymer LD/talc mixture used in this Example was 3.5 μm and D90 was 10.2 μm. As shown in Table 3, the amount of coating was selected as 25% by weight or 43% by weight with respect to a nuclear particle to be coated.

First, a mixture of dried methacrylic acid copolymer LD and talc (mass ratio: dried methacrylic acid copolymer LD:talc=2:1) was pulverized with a spiral jet mill (100 AS, Hosokawa Micron Corporation) to obtain particle mixture for coating 3. The mean particle size (D50) of the mixture at this time was about 3.5 μm, and the D90 was about 10.2 μm. Examples 2-1 and 2-2 were manufactured in accordance with the formulation ratio and amount described in Table 3. Specifically, compound A and granularity controlled product of aminoalkyl methacrylate copolymer RS (No. 100 ON fraction) were loaded into a high speed stirring granulator (model FM-VG-05, capacity: 5L, Powrex Corp) and granulated while spraying a suitable amount of aqueous 95% ethanol solution under the mixing and granulating conditions shown in Table 2 to obtain nuclear particles to be coated in a wet powder state. The nuclear particles to be coated in a wet powder state were loaded into a fluidized bed dryer (MP-01, Powrex Corp) and dried under the drying conditions shown in Table 2 to obtain nuclear particles to be coated. The nuclear particles to be coated were loaded into a high-speed stirring granulator, i.e., Vertical Granulator (model FM-VG-01, Powrex Corp), and coated while spraying the aqueous 95% ethanol solution described in Table 3 under the mixing/coating conditions specified in Table 2 as particle mixture for coating 2 was separated and added 2 times at 28 g, 3 times at 23 g, and 3 times at 30 g. When particle mixture for coating 3 was added to an amount equivalent to 20% by weight (when 100 g of particle mixture for coating was added and coated), samples were collected. The sampled component of interest-containing hollow particles in a wet powder state were loaded into a rack dryer (Perfect Oven, ESPEC Corp.) and dried overnight at 50° C. to obtain the component of interest-containing hollow particles of Example 2-1. Subsequent to the sampling, the coating step was continued until 200 g of mixture for coating 3 was coated to obtain component of interest-containing hollow particles in a wet powder state. The component of interest-containing hollow particles in a wet powder state were loaded into a rack dryer (Perfect Oven, ESPEC Corp.) and dried overnight at 50° C. to obtain the component of interest-containing hollow particles of Example 2-2.

TABLE 3

| | Example 2-1 | | Example 2-2 | |
|---|---|---|---|---|
| | Amount | Formulation | Amount | Formulation |
| | loaded (g) | ratio (%) | loaded (g) | ratio (%) |
| Compound A | 400 | 64.0 | 400 | 55.9 |
| (ground | | | | |
| product of JM) | | | | |
| Aminoalkyl | 100 | 16.0 | 100 | 14.0 |
| methacrylate | | | | |
| copolymer | | | | |
| RS (No. 100 on) | | | | |
| Particle mixture for | 125 | 20.0 | 215 | 30.1 |
| coating 3 (dried | | | | |
| methacrylic acid | | | | |
| copolymer | | | | |
| LD: talc = 2:1) | | | | |
| (Aqueous 95% | (55) | (8.8) | (100) | (14.0) |
| ethanol solution) | | | | |
| (for coating) | | | | |
| Total | 625 | 100 | 715 | 100 |

### Test Example 2<Dissolution Test on Component of Interest-Containing Hollow Particles with Different Coatable Microparticle Sizes>

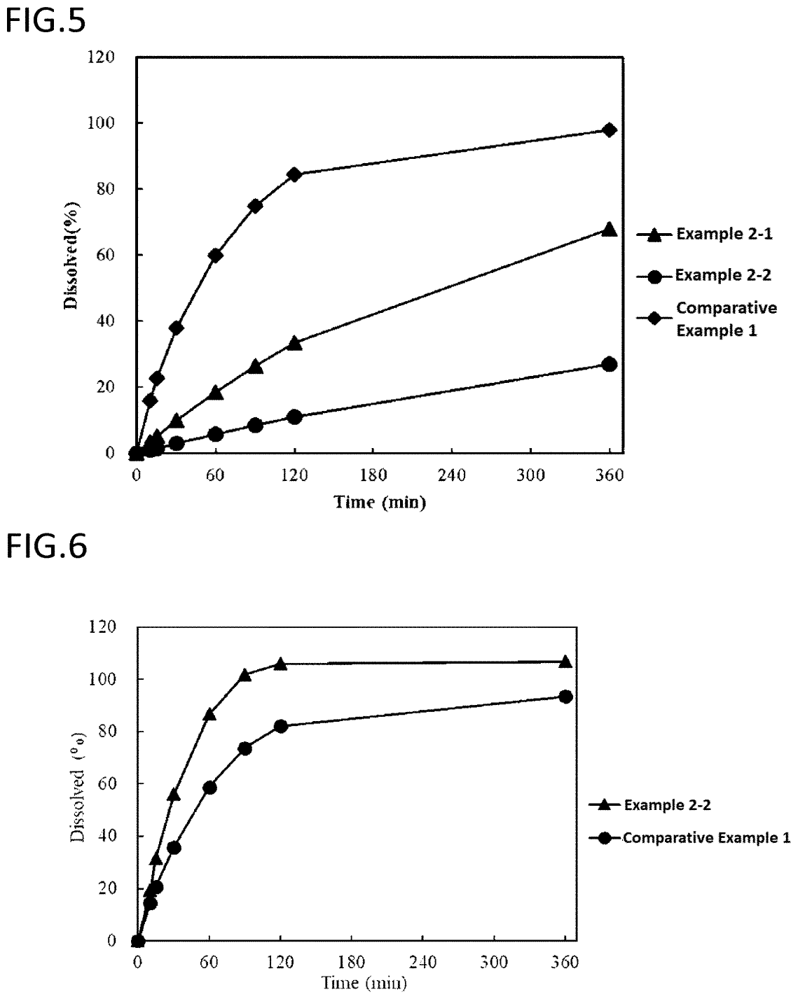

Dissolution tests were conducted using the particles manufactured in Example 2. The test conditions were the same as in Test Example 1. The results are shown in FIGS. 5 and 6. FIG. 5 is the test result using 1st fluid for dissolution test, and FIG. 6 is the test result using 2nd fluid for dissolution test. Table 6 shows the coating time and the time that was required for the manufacture of the resulting particles.

Table 6 shows the ratio of 50% dissolution times before and after coating for the component of interest-containing hollow particles using coatable microparticle of all particle sizes. An effect of suppressing the release rate was attained.

### Example 3<Manufacture of Component of Interest-Containing Hollow Particles Using Coatable Microparticle with Different Types of Deflocculating Agent (Lubricant)>

Example 3 manufactured the component of interest-containing hollow particles of the present disclosure with different deflocculating agent (lubricant) constituting coatable microparticle.

As the deflocculating agent (lubricant), sodium stearyl fumarate and titanium oxide were used. As shown in Table 4, the amount of coating was selected as 20% by weight or 40% by weight with respect to a nuclear particle to be coated.

First, 133.4 g of particle mixture for coating 1, which is a pulverized product of the mixture of dried methacrylic acid copolymer LD and magnesium aluminosilicate manufactured in Example 1 (mass ratio: dried methacrylic acid copolymer LD:magnesium aluminosilicate=1:0.05), was mixed with 66.6 g of sodium stearyl fumarate or titanium oxide to obtain particle mixture for coating 4 and particle mixture for coating 5, respectively. The mean particle sizes (D50) of the sodium stearyl fumarate and titanium oxide at this time were about 9.6 μm and about 6.9 μm, and the D90 was about 22.8 μm and about 19.8 μm, respectively. Examples 3-1 to 3-4 were manufactured in accordance with the formulation ratio and amount described in Table 4. Specifically, compound A and granularity controlled product of aminoalkyl methacrylate copolymer RS (No. 100 ON fraction) were loaded into a high speed stirring granulator (model FM-VG-05, capacity: 5L, Powrex Corp) and granulated while spraying a suitable amount of aqueous 95% ethanol solution under the mixing and granulating conditions shown in Table 2 to obtain nuclear particles to be coated in a wet powder state. The nuclear particles to be coated in a wet powder state were loaded into a fluidized bed dryer (MP-01, Powrex Corp) and dried under the drying conditions shown in Table 2 to obtain nuclear particles to be coated. The nuclear particles to be coated were loaded into a high-speed stirring granulator, i.e., Vertical Granulator (model FM-VG-01, Powrex Corp), and coated while spraying the aqueous 95% ethanol solution described in Table 4 under the mixing/coating conditions specified in Table 2 as particle mixture for coating 4 or 5 was separated and added 8 times at 25 g each. When particle mixture for coating 4 or 5 was added to an amount equivalent to 20% by weight (when 100 g of particle mixture for coating was added and coated), samples were collected. The sampled component of interest-containing hollow particles in a wet powder state were loaded into a rack dryer (Perfect Oven, ESPEC Corp.) and dried overnight at 50° C. to obtain the component of interest-containing hollow particles of Example 3-1 or 3-3. Subsequent to the sampling, the coating step was continued until 200 g of mixture for coating 4 or 5 was coated to obtain component of interest-containing hollow particles in a wet powder state. The component of interest-containing hollow particles in a wet powder state were loaded into a rack dryer (Perfect Oven, ESPEC Corp.) and dried overnight at 50° C. to obtain the component of interest-containing hollow particles of Example 3-2 or 3-4.

TABLE 4

| | Example 3-1 | | Example 3-2 | | Example 3-3 | | Example 3-4 | |
|---|---|---|---|---|---|---|---|---|
| | Amount | Formulation | Amount | Formulation | Amount | Formulation | Amount | Formulation |
| | loaded | ratio | loaded | ratio | loaded | ratio | loaded | ratio |
| | (g) | (%) | (g) | (%) | (g) | (%) | (g) | (%) |
| Compound A | 400 | 66.7 | 400 | 57.1 | 400 | 66.7 | 400 | 57.1 |
| (ground product | | | | | | | | |
| of JM) | | | | | | | | |
| Aminoalkyl | 100 | 16.7 | 100 | 14.3 | 100 | 16.7 | 100 | 14.3 |
| methacrylate | | | | | | | | |
| copolymer RS | | | | | | | | |
| (No. 100 on) | | | | | | | | |
| Particle mixture | 66.7 | 11.1 | 133.4 | 19.1 | 66.7 | 11.1 | 133.4 | 19.1 |
| for coating 1 | | | | | | | | |
| (dried | | | | | | | | |
| methacrylic acid | | | | | | | | |
| copolymer | | | | | | | | |
| LD:magnesium | | | | | | | | |
| aluminosilicate = | | | | | | | | |
| 1:0.05) | | | | | | | | |
| Titanium oxide | 33.3 | 5.6 | 66.6 | 9.5 | — | — | — | — |
| Sodium stearyl | — | — | — | — | 33.3 | 5.6 | 66.6 | 9.5 |
| fumarate | | | | | | | | |
| (Aqueous 95% | (68) | (11.3) | (127) | (18.1) | (65) | (10.8) | (137) | (19.6) |
| ethanol solution) | | | | | | | | |
| (for coating) | | | | | | | | |
| Total | 600 | 100 | 700 | 100 | 600 | 100 | 700 | 100 |

### Test Example 3<Dissolution Test on Component of Interest-Containing Hollow Particles with a Coatable First Microparticle and a Lubricant of Different Particle Sizes>

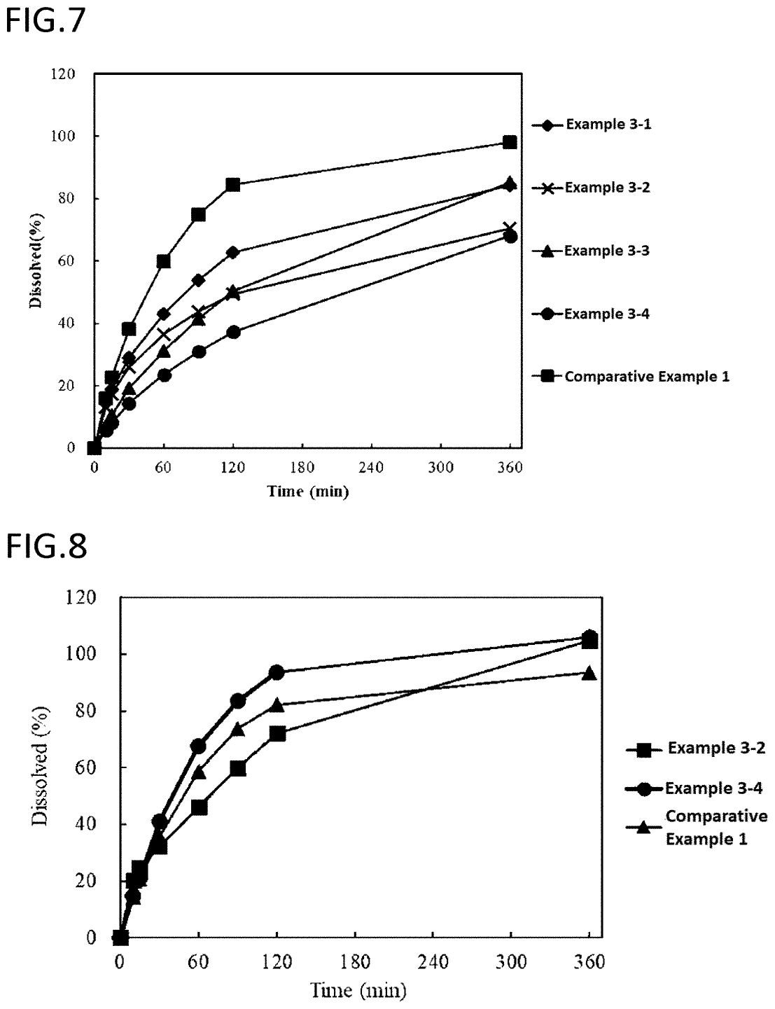

Dissolution tests were conducted using the particles manufactured in Example 3. The test conditions were the same as in Test Example 1. The results are shown in FIGS. 7 and 8. Table 6 shows the coating time and the time that was required for the manufacture of the resulting particles. FIG. 7 is the test result using 1st fluid for dissolution test, and FIG. 8 is the test result using 2nd fluid for dissolution test.

Table 6 shows the ratio of 50% dissolution times before and after coating for the component of interest-containing hollow particles using a coatable powdered first microparticle and a lubricant of all particle sizes. An effect of suppressing the release rate was attained.

### Example 4<Manufacture of Component of Interest-Containing Hollow Particles Using Coatable Microparticle with Different Ratios of a First Macromolecule to a Lubricant>

Example 4 manufactured the component of interest-containing hollow particles of the present disclosure with different ratios of a coatable first macromolecule to a lubricant.

As the deflocculating agent (lubricant), talc was used. The ratio of a first macromolecule to a lubricant was set to a ratio of 1:0.25 or 1:2. As shown in Table 5, the amount of coating was selected as 20% by weight or 40% by weight with respect to a nuclear particle.

First, 66.6 g of particle mixture for coating 1, which is a pulverized product of the mixture of dried methacrylic acid copolymer LD and magnesium aluminosilicate manufactured in Example 1 (mass ratio: dried methacrylic acid copolymer LD:magnesium aluminosilicate=1:0.05), was mixed with 133.4 g of talc as particle mixture for coating 6, and 160 g of particle mixture for coating 1 was mixed with 40 g of talc as particle mixture for coating 7. Examples 4-1 to 4-4 were manufactured in accordance with the formulation ratio and amount described in Table 5. Specifically, compound A and granularity controlled product of aminoalkyl methacrylate copolymer RS (No. 100 ON fraction) were loaded into a high speed stirring granulator (model FM-VG-05, capacity: 5L, Powrex Corp) and granulated while spraying a suitable amount of aqueous 95% ethanol solution under the mixing and granulating conditions shown in Table 2 to obtain nuclear particles to be coated in a wet powder state. The nuclear particles in a wet powder state were loaded into a fluidized bed dryer (MP-01, Powrex Corp) and dried under the drying conditions shown in Table to obtain nuclear particles to be coated. The nuclear particles to be coated were loaded into a high-speed stirring granulator, i.e., Vertical Granulator (model FM-VG-01, Powrex Corp), and coated while spraying the aqueous 95% ethanol solution described in Table 5 under the mixing/coating conditions specified in Table 2 as particle mixture for coating 6 or 7 was separated and added 8 times at 25 g each. When particle mixture for coating 6 or 7 was added to an amount equivalent to 20% by weight (when 100 g of particle mixture for coating was added and coated), samples were collected. The sampled component of interest-containing hollow particles in a wet powder state were loaded into a rack dryer (Perfect Oven, ESPEC Corp.) and dried overnight at 50° C. to obtain the component of interest-containing hollow particles of Example 4-1 or 4-3. Subsequent to the sampling, the coating step was continued until 200 g of mixture for coating 6 or 7 was coated to obtain component of interest-containing hollow particles in a wet powder state. The component of interest-containing hollow particles in a wet powder state were loaded into a rack dryer (Perfect Oven, ESPEC Corp.) and dried overnight at 50° C. to obtain the component of interest-containing hollow particles of Example 4-2 or 4-4.

TABLE 5

| | Example 4-1 | | Example 4-2 | | Example 4-3 | | Example 4-4 | |
|---|---|---|---|---|---|---|---|---|
| | Amount | Formulation | Amount | Formulation | Amount | Formulation | Amount | Formulation |
| | loaded | ratio | loaded | ratio | loaded | ratio | loaded | ratio |
| | (g) | (%) | (g) | (%) | (g) | (%) | (g) | (%) |
| Compound A | 400 | 66.7 | 400 | 57.1 | 400 | 66.7 | 400 | 57.1 |
| (ground product | | | | | | | | |
| of JM) | | | | | | | | |
| Aminoalkyl | 100 | 16.7 | 100 | 14.3 | 100 | 16.7 | 100 | 14.3 |
| methacrylate | | | | | | | | |
| copolymer RS | | | | | | | | |
| (No. 100 on) | | | | | | | | |
| Particle mixture | 33.3 | 5.6 | 66.6 | 9.5 | 80 | 13.3 | 160 | 22.9 |
| for coating 1 | | | | | | | | |
| (dried | | | | | | | | |
| methacrylic acid | | | | | | | | |
| copolymer | | | | | | | | |
| LD:magnesium | | | | | | | | |
| aluminosilicate = | | | | | | | | |
| 1:0.05) | | | | | | | | |
| Talc | 66.7 | 11.1 | 133.4 | 19.1 | 20 | 3.3 | 40 | 5.7 |
| (Aqueous 95% | (68) | (11.3) | (151) | (21.6) | (91) | (15.2) | (245) | (35.0) |
| ethanol solution) | | | | | | | | |
| (for coating) | | | | | | | | |
| Total | 600 | 100 | 700 | 100 | 600 | 100 | 700 | 100 |

### Test Example 4<Dissolution Test on Component of Interest-Containing Hollow Particles with Different Coatable Microparticle Sizes>

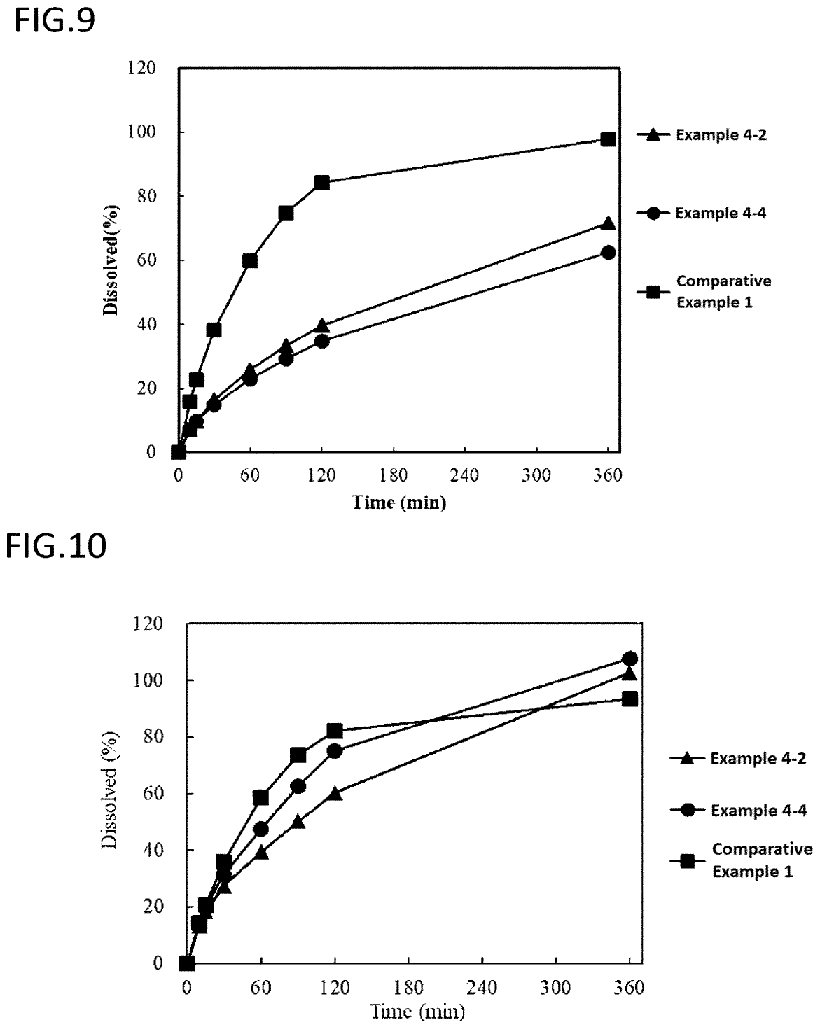

Dissolution tests were conducted using the particles manufactured in Example 4. The test conditions were the same as in Test Example 1. The results are shown in FIGS. 9 and 10. Table 6 shows the coating time and the time that was required for the manufacture of the resulting particles. FIG. 9 is the test result using 1st fluid for dissolution test, and FIG. 10 is the test result using 2nd fluid for dissolution test.

Table 6 shows the ratios of 50% dissolution times before and after coating for the component of interest-containing hollow particles using coatable microparticle of all particle sizes. An effect of suppressing the release rate was attained.

TABLE 6

| | 50% | Ratio of 50% dissolution | Manufacturing | Amount of |
|---|---|---|---|---|
| | dissolution | times with respect to | time (coating) | solvent used |
| | time | nuclear particles | (minutes) | (g) |
| Comparative | 46.4 | 1 | — | — |
| Example 1 | | | | |
| Example 1-1 | 83 | 1.8 | 18.75 | 55 |
| Example 1-2 | 327.8 | 7.1 | 35.25 | 110 |
| Example 2-1 | 234.1 | 5 | 34.5 | 114 |
| Example 2-2 | 941.4 | 20.3 | 62.5 | 201 |
| Example 3-1 | 79.2 | 1.7 | 28.25 | 68 |
| Example 3-2 | 128 | 2.8 | 62.75 | 127 |
| Example 3-3 | 119 | 2.6 | 29 | 65 |
| Example 3-4 | 219.3 | 4.7 | 66.5 | 137 |
| Example 4-1 | 145.6 | 3.1 | 43 | 68 |
| Example 4-2 | 197.5 | 4.3 | 70.25 | 151 |
| Example 4-3 | 90 | 1.9 | 61.5 | 91 |
| Example 4-4 | 251.6 | 5.4 | 114.5 | 245 |

### Example 5<Manufacture of Component of Interest-Containing Hollow Particles Using an Insoluble Macromolecule Particle as a First Macromolecule and a Stomach Soluble Macromolecule Particle as a Second Macromolecule>

Example 5 manufactured component of interest-containing hollow particles using an insoluble macromolecule particle as a first macromolecule and a stomach soluble macromolecule particles as a second macromolecule.

As the deflocculating agent (lubricant), talc was used. As shown in Table 7, the amount of coating was selected as 20% by weight or 40% by weight with respect to a nuclear particle to be coated. As antistatic agents, Neusilin UFL2 was used.

First, aminoalkyl methacrylate copolymer E100 was pulverized with a fitzmill DKA-6 (Hosokawa Micron Corporation). The pulverized aminoalkyl methacrylate copolymer E100 was passed through a No. 100 mesh sieve, and the residual on the sieve was used as aminoalkyl methacrylate copolymer E (No. 100 on). 40 g of a water insoluble macromolecule Ethocel 10FP mixed with 20 g of talc was prepared as particle mixture for coating 8. The mean particle size (D50) of particle mixture for coating 8 was about 4.7 μm, and D90 was about 9.1 μm. The mean particle size (D50) of Ethocel 10FP was about 5.0 μm, and D90 was about 9.1 μm.

Examples 5-1 to 5-2 were manufactured in accordance with the formulation ratio and amount described in Table 7. Specifically, compound A and granularity controlled product of pulverized aminoalkyl methacrylate copolymer E100 (No. 100 ON fraction) were loaded into a container rotating granulator, Intensive Mixer (EL-1, Nippon Eirich) and granulated while spraying a suitable amount of aqueous 95% ethanol solution under the mixing and granulating conditions shown in Table 8 to obtain nuclear particles to be coated in a wet powder state. The nuclear particles in a wet powder state were loaded into a fluidized bed dryer (MP-01, Powrex Corp) and dried under the drying conditions shown in Table 2 to obtain nuclear particles to be coated. The nuclear particles to be coated were loaded into a container rotating granulator, Intensive Mixer (EL-1, Nippon Eirich), and coated while spraying the aqueous 95% ethanol solution described in Table 7 under the mixing/coating conditions specified in Table 8 as particle mixture for coating 8 was separated and added 8 times at 7.5 g each. When particle mixture for coating 8 was added to an amount equivalent to 20% by weight (when 30 g of particle mixture for coating was added and coated), samples were collected. The sampled component of interest-containing hollow particles in a wet powder state were loaded into a rack dryer (Perfect Oven, ESPEC Corp.) and dried for 2 hours at 60° C. to obtain the component of interest-containing hollow particles of Example 5-1. Subsequent to the sampling, the coating step was continued until 60 g of mixture for coating 8 was coated to obtain component of interest-containing hollow particles in a wet powder state. The component of interest-containing hollow particles in a wet powder state were loaded into a fluidized bed dryer (MP-01, Powrex Corp) and dried under the drying conditions shown in Table 2. After drying, Neusilin was added and mixed within the fluidized bed granulator MP-01 to obtain the component of interest-containing hollow particles of Example 5-2.

TABLE 7

| | Comparative Example 5 | | Example 5-1 | | Example 5-2 | |
|---|---|---|---|---|---|---|
| | Amount | Formulation | Amount | Formulation | Amount | Formulation |
| | loaded | ratio | loaded | ratio | loaded | ratio |
| | (g) | (%) | (g) | (%) | (g) | (%) |
| Compound A | 160 | 80 | 120 | 66.7 | 120 | 56.9 |
| (ground product | | | | | | |
| of JM) | | | | | | |
| Aminoalkyl | 40 | 20 | 30 | 16.7 | 30 | 14.2 |
| methacrylate | | | | | | |
| copolymer E | | | | | | |
| (No. 100 on) | | | | | | |
| Ethocel 10FP | — | — | 20 | 11.1 | 40 | 19.0 |
| Talc | — | — | 10 | 5.6 | 20 | 9.5 |
| Neusilin UFL2 | — | — | — | — | 1 | 0.5 |
| Aqueous 95% | 32 | 16 | — | — | — | — |
| ethanol solution | | | | | | |
| (for nuclear | | | | | | |
| particle) | | | | | | |
| Aqueous 95% | — | — | 30 | 16.7 | 54 | 25.6 |
| ethanol solution | | | | | | |
| (for coating) | | | | | | |
| Total | 200 | 100 | 180 | 100 | 211 | 100 |

TABLE 8

| | | | | Number | | | |
|---|---|---|---|---|---|---|---|
| | | | Rotor | of | Container | Container | |
| | Equipment | Mixing | rotation | rotations | rotation | rotation | Container |
| | name | time | direction | of rotor | direction | rate | angle |
| Premixing | Intensive | 1 min | Clockwise | 2450 min⁻¹ | Clockwise | Low speed | 30° |
| step | mixer (EL- | | rotation | | rotation | | |
| | 1, Nippon | | | | | | |
| | Eirich) | | | | | | |

| | | | Number | | | | | | |
|---|---|---|---|---|---|---|---|---|---|
| | | Rotor | of | Container | Container | | Liquid | | |
| | Equipment | rotation | rotations | rotation | rotation | Container | addition | Spray | Spray |
| | name | direction | of rotor | direction | rate | angle | method | pressure | rate |
| Granulation | Intensive | Clockwise | 2450 min⁻¹ | Clockwise | Low speed | 30° | Spray | 0.08 MPa | 4 g/min |
| step | mixer (EL- | rotation | | rotation | | | | | |
| | 1, Nippon | | | | | | | | |
| | Eirich) | | | | | | | | |
| | | | Number | | | | | | |
| | | Rotor | of | Container | Container | | Liquid | | |
| | Equipment | rotation | rotations | rotation | rotation | Container | addition | Spray | Spray |
| | name | direction | of rotor | direction | rate | angle | method | pressure | rate |
| Coating | Intensive | Clockwise | 2450 min⁻¹ | Clockwise | Low speed | 30° | Spray | 0.08 MPa | 4 g/min |
| step | mixer (EL- | rotation | | rotation | | | | | |
| | 1, Nippon | | | | | | | | |
| | Eirich) | | | | | | | | |

### Comparative Example 5 Manufacture of Nuclear Particles to be Coated

In Comparative Example 5, only particles that were not coated, i.e., nuclear particles to be coated, were manufactured in accordance with the formulation ratio and amount loaded described in Table 7 in the same manner as Example 5. After granulating nuclear particles to be coated in a wet powder state as in Example 5, the nuclear particles to be coated in a wet powder state were subjected to fluidized bed drying using a fluidized bed dryer (MP-01, Powrex Corp) to obtain the nuclear particles to be coated of Comparative Example 5.

### Test Example 5<Dissolution Test on Component of Interest-Containing Hollow Particles Using a Water Insoluble Macromolecule Particle as a First Macromolecule and a Stomach Soluble Macromolecule Particle as a Second Macromolecule>

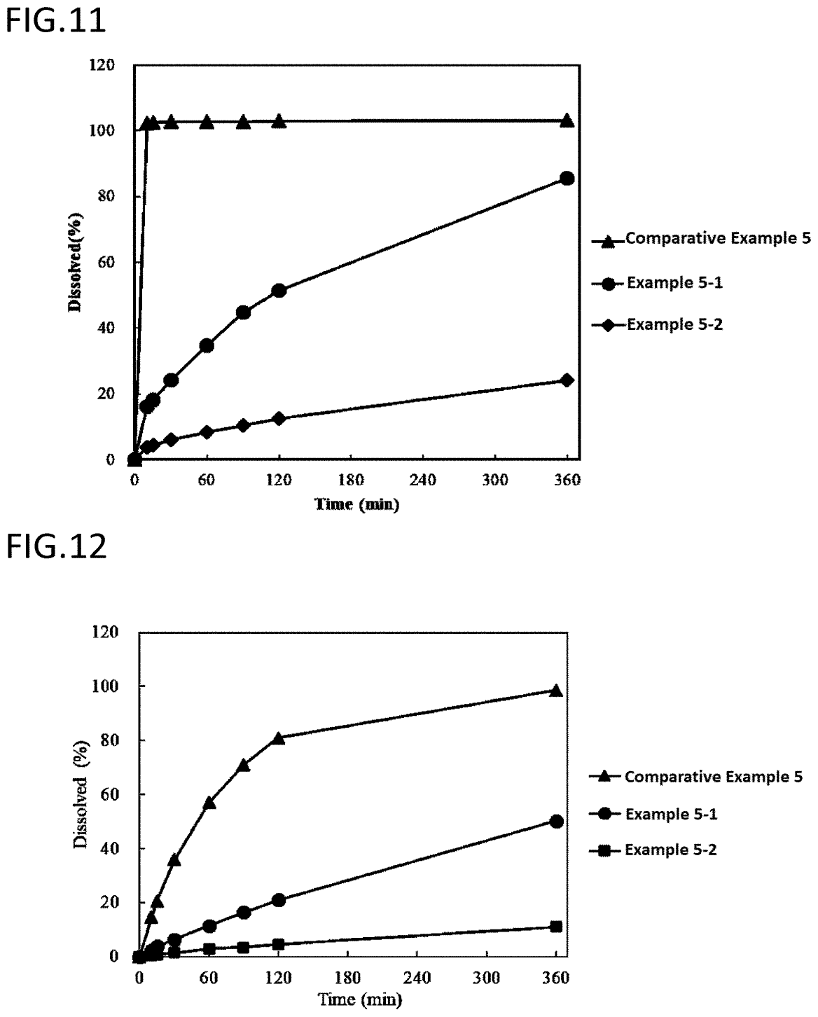

Dissolution tests were conducted using the particles manufactured in Example 5. The test conditions were the same as Test Example 1. The results are shown in FIGS. 11 and 12. FIG. 11 is the test result using 1st fluid for dissolution test, and FIG. 12 is the test result using 2nd fluid for dissolution test. Table 12 shows the coating time and the time that was required for the manufacture of the resulting particles.

Tables 12 and 13 show the ratios of 50% dissolution times before and after coating for the component of interest-containing hollow particles. An effect of suppressing the release rate was attained.

### Example 6<Manufacture of Component of Interest-Containing Hollow Particles Using a Water Insoluble Macromolecule Particle as a First Macromolecule and a Water Soluble Macromolecule Particle as a Second Macromolecule>

Example 6 manufactured component of interest-containing hollow particles using a water insoluble macromolecule particle as a first macromolecule and a water soluble macromolecule particle as a second macromolecule.

As the deflocculating agent (lubricant), talc was used. As shown in Table 7, the amount of coating was selected as 20% by weight or 40% by weight with respect to a nuclear particle to be coated. As antistatic agents, Neusilin UFL2 was used.

40 g of a water insoluble macromolecule Ethocel 10FP mixed with 20 g of talc was prepared as particle mixture for coating 8. Hydroxypropyl cellulose was passed through a No. 100 sieve, and the residual on the sieve was used as hydroxypropyl cellulose (No. 100 on).

Examples 6-1 to 6-2 were manufactured in accordance with the formulation ratio and amount described in Table 9. Specifically, compound A and granularity controlled product of hydroxypropyl cellulose (No. 100 ON fraction) were loaded into a container rotating granulator, Intensive Mixer (EL-1, Nippon Eirich) and granulated while spraying a suitable amount of aqueous 95% ethanol solution under the mixing and granulating conditions shown in Table 8 to obtain nuclear particles to be coated in a wet powder state. The nuclear particles in a wet powder state were loaded into a fluidized bed dryer (MP-01, Powrex Corp) and dried under the drying conditions shown in Table 2 to obtain nuclear particles to be coated. The nuclear particles to be coated were loaded into a container rotating granulator, Intensive Mixer (EL-1, Nippon Eirich), and coated while spraying the aqueous 95% ethanol solution described in Table 7 under the coating conditions specified in Table 8 as particle mixture for coating 8 was separated and added 8 times at 7.5 g each. When particle mixture for coating 8 was added to an amount equivalent to 20% by weight (when 30 g of particle mixture for coating was added and coated), samples were collected. The sampled component of interest-containing hollow particles in a wet powder state were loaded into a rack dryer (Perfect Oven, ESPEC Corp.) and dried for 2 hours at 60° C. to obtain the component of interest-containing hollow particles of Example 6-1. Subsequent to the sampling, the coating step was continued until 60 g of mixture for coating 8 was coated to obtain component of interest-containing hollow particles in a wet powder state. The component of interest-containing hollow particles in a wet powder state were loaded into a fluidized bed dryer (MP-01, Powrex Corp) and dried under the drying conditions shown in Table 2. After drying, Neusilin was added and mixed within the fluidized bed granulator MP-01 to obtain the component of interest-containing hollow particles of Example 6-2.

TABLE 9

| | Comparative Example 6 | | Example 6-1 | | Example 6-2 | |
|---|---|---|---|---|---|---|
| | Amount | Formulation | Amount | Formulation | Amount | Formulation |
| | loaded | ratio | loaded | ratio | loaded | ratio |
| | (g) | (%) | (g) | (%) | (g) | (%) |
| Compound A | 160 | 80 | 120 | 66.7 | 120 | 56.9 |
| (ground product | | | | | | |
| of JM) | | | | | | |
| Hydroxypropyl | 40 | 20 | 30 | 16.7 | 30 | 14.2 |
| cellulose | | | | | | |
| (No. 100 on) | | | | | | |
| Ethocel 10FP | — | — | 20 | 11.1 | 40 | 19.0 |
| Talc | — | — | 10 | 5.6 | 20 | 9.5 |
| Neusilin UFL2 | — | — | — | — | 1 | 0.5 |
| Aqueous 95% | 36 | 18 | — | — | — | — |
| ethanol solution | | | | | | |
| (for nuclear | | | | | | |
| particle) | | | | | | |
| Aqueous 95% | — | — | 32 | 17.8 | 60 | 28.4 |
| ethanol solution | | | | | | |
| (for coating) | | | | | | |
| Total | 200 | 100 | 180 | 100 | 211 | 100 |

### Comparative Example 6 Manufacture of Nuclear Particles to be Coated

In Comparative Example 6, only particles that were not coated, i.e., nuclear particles to be coated, were manufactured in accordance with the formulation ratio and amount loaded described in Table 9 in the same manner as Example 6. After granulating nuclear particles to be coated in a wet powder state as in Example 6, the nuclear particles to be coated in a wet powder state were subjected to fluidized bed drying using a fluidized bed dryer (model MP-01, Powrex Corp) to obtain the nuclear particles to be coated of Comparative Example 6.

### <Test Example 6> <Dissolution Test on Component of Interest-Containing Hollow Particles Using a Water Soluble Macromolecule Particle as a First Macromolecule and a Water Insoluble Macromolecule Particle as a Second Macromolecule>

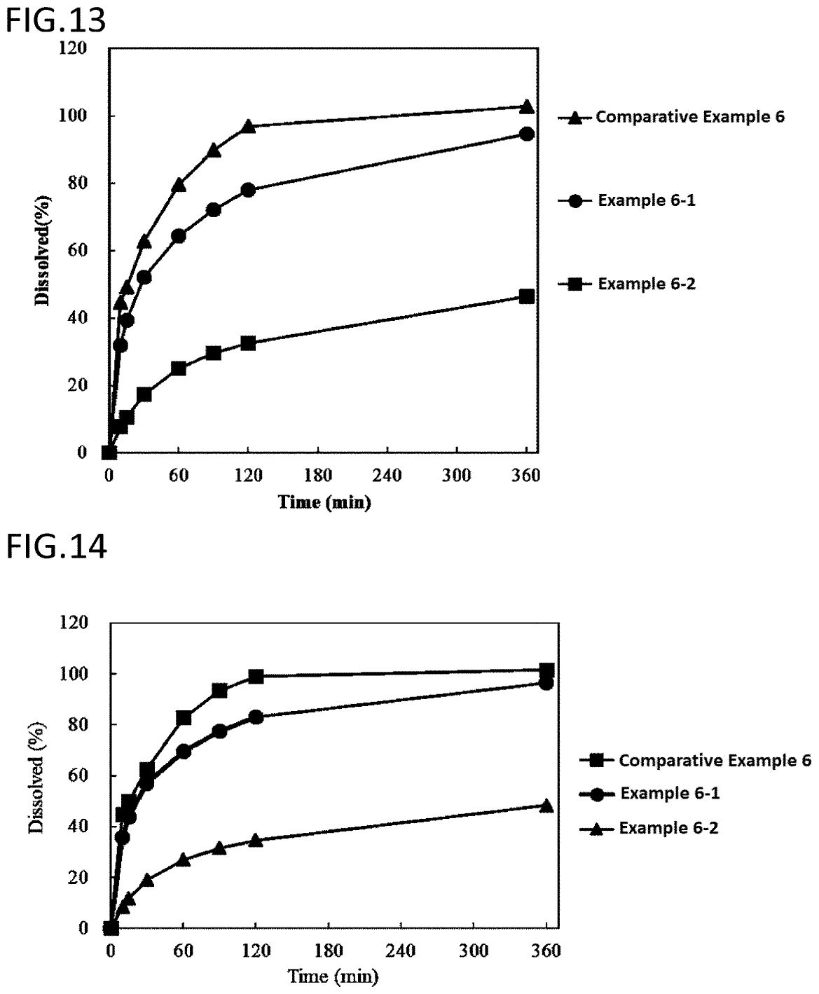

Dissolution tests were conducted using the particles manufactured in Example 6. The test conditions were the same as Test Example 1. The results are shown in FIGS. 13 and 14. FIG. 13 is the test result using 1st fluid for dissolution test, and FIG. 14 is the test result using 2nd fluid for dissolution test. Table 12 shows the coating time and the time that was required for the manufacture of the resulting particles.

Tables 12 and 13 show the ratios of 50% dissolution times before and after coating for the component of interest-containing hollow particles. An effect of suppressing the release rate was attained.

### Example 7<Manufacture of Hollow Particles Containing Compound B as a Component of Interest Using a Water Insoluble Macromolecule Particle as a First Macromolecule and a Stomach Soluble Macromolecule Particle as a Second Macromolecule>

Example 7 manufactured hollow particles containing compound B as a component of interest using a water insoluble macromolecule particle as a first macromolecule and a stomach soluble macromolecule particle as a second macromolecule.

As the deflocculating agent (lubricant), talc was used. As shown in Table 7, the amount of coating was selected as 20% by weight or 40% by weight with respect to a nuclear particle to be coated. As antistatic agents, Neusilin UFL2 was used.

40 g of a water insoluble macromolecule Ethocel 10FP mixed with 20 g of talc was prepared as particle mixture for coating 8. The aminoalkyl methacrylate copolymer E100 (No. 100 on) prepared in Example 5 was also used.

Examples 7-1 to 7-2 were manufactured in accordance with the formulation ratio and amount described in Table 10. Specifically, compound B and granularity controlled product of aminoalkyl methacrylate copolymer E100 (No. 100 ON fraction) were loaded into a container rotating granulator, Intensive Mixer (EL-1, Nippon Eirich) and granulated while spraying a suitable amount of aqueous 95% ethanol solution under the mixing and granulating conditions shown in Table 8 to obtain nuclear particles to be coated in a wet powder state. The nuclear particles in a wet powder state were loaded into a fluidized bed dryer (MP-01, Powrex Corp) and dried under the drying conditions shown in Table 2 to obtain nuclear particles to be coated. The nuclear particles to be coated were loaded into a container rotating granulator, Intensive Mixer (EL-1, Nippon Eirich), and coated while spraying the aqueous 95% ethanol solution described in Table 7 under the coating conditions specified in Table 8 as particle mixture for coating 8 was separated and added 8 times at 7.5 g each. When particle mixture for coating 8 was added to an amount equivalent to 20% by weight (when 30 g of particle mixture for coating was added and coated), samples were collected. The sampled component of interest-containing hollow particles in a wet powder state were loaded into a rack dryer (Perfect Oven, ESPEC Corp.) and dried for 2 hours at 60° C. to obtain the component of interest-containing hollow particles of Example 7-1. Subsequent to the sampling, the coating step was continued until 60 g of mixture for coating 8 was coated to obtain component of interest-containing hollow particles in a wet powder state. The component of interest-containing hollow particles in a wet powder state were loaded into a fluidized bed dryer (MP-01, Powrex Corp) and dried under the drying conditions shown in Table 2. After drying, Neusilin was added and mixed within the fluidized bed granulator MP-01 to obtain the component of interest-containing hollow particles of Example 7-2.

TABLE 10

| | Comparative Example 7 | | Example 7-1 | | Example 7-2 | |
|---|---|---|---|---|---|---|
| | Amount | Formulation | Amount | Formulation | Amount | Formulation |
| | loaded | ratio | loaded | ratio | loaded | ratio |
| | (g) | (%) | (g) | (%) | (g) | (%) |
| Compound B | 160 | 80 | 120 | 66.7 | 120 | 56.9 |
| (ground product | | | | | | |
| of JM) | | | | | | |
| Aminoalkyl | 40 | 20 | 30 | 16.7 | 30 | 14.2 |
| methacrylate | | | | | | |
| copolymer E | | | | | | |
| (No. 100 on) | | | | | | |
| Ethocel 10FP | — | — | 20 | 11.1 | 40 | 19.0 |
| Talc | — | — | 10 | 5.6 | 20 | 9.5 |
| Neusilin UFL2 | — | — | — | — | 1 | 0.5 |
| Aqueous 95% | 40 | 20 | — | — | — | — |
| ethanol solution | | | | | | |
| (for nuclear | | | | | | |
| particle) | | | | | | |
| Aqueous 95% | — | — | 28 | 15.6 | 46 | 21.8 |
| ethanol solution | | | | | | |
| (for coating) | | | | | | |
| Total | 200 | 100 | 180 | 100 | 211 | 100 |

### Comparative Example 7 Manufacture of Nuclear Particles to be Coated

In Comparative Example 7, only particles that were not coated, i.e., nuclear particles to be coated, were manufactured in accordance with the formulation ratio and amount loaded described in Table 10 in the same manner as Example 7. After granulating nuclear particles to be coated in a wet powder state as in Example 7, the nuclear particles to be coated in a wet powder state were subjected to fluidized bed drying using a fluidized bed dryer (model MP-01, Powrex Corp) to obtain the nuclear particles to be coated of Comparative Example 7.

### <Test Example 7> <Dissolution Test on Hollow Particles Containing Compound B as a Component of Interest Using a Water Insoluble Macromolecule Particle as a First Macromolecule and a Stomach Soluble Macromolecule Particle as a Second Macromolecule>

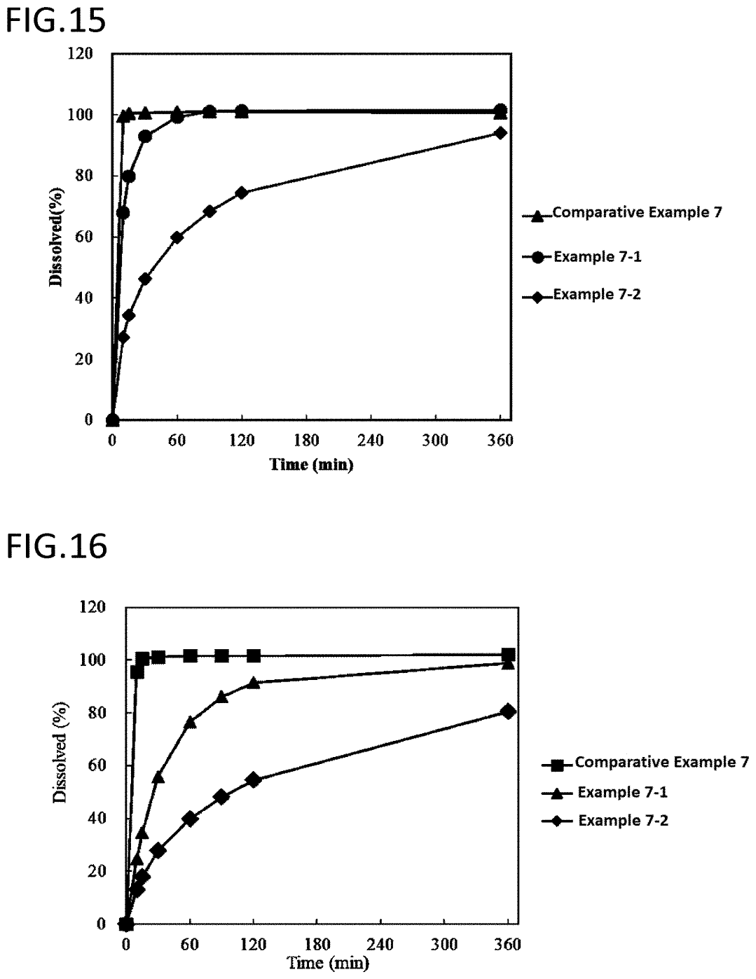

Dissolution tests were conducted using the particles manufactured in Example 7. The dissolution test conditions were the same as in Test Example 1. HPLC measurement conditions are shown below. The results are shown in FIGS. 15 and 16. FIG. 15 is the test result using 1st fluid for dissolution test, and FIG. 16 is the test result using 2nd fluid for dissolution test. Table 12 shows the coating time and the time that was required for the manufacture of the resulting particles.

<HPLC Measurement Conditions>

### HPLC: UFLC XR/Shimadzu Corporation (LC-208)

Detector: UV detector
Measurement wavelength: 244 nm
Column: XBridge C18 (4.6 mm×100 mm, 3.5 μm)
Column temperature: 40° C.
Mobile phase: 0.01 mol/L phosphate buffer (pH 6.8)/methanol mixture (8:2)
Mobile phase flow rate: 1.0 mL/min
Injection volume: 5 μL
Sample cooler temperature: 25° C.
Syringe cleansing solution: water/methanol mixture=(3:7)

Tables 12 and 13 show the ratios of 50% dissolution times before and after coating for the component of interest-containing hollow particles. An effect of suppressing the release rate was attained.

### Example 8<Manufacture of Hollow Particles Containing Compound C as a Component of Interest Using a Water Insoluble Macromolecule Particle as a First Macromolecule and a Stomach Soluble Macromolecule Particle as a Second Macromolecule>

Example 8 manufactured hollow particles containing compound C as a component of interest using a water insoluble macromolecule particles as a first macromolecule and a stomach soluble macromolecule particle as a second macromolecule.

As the deflocculating agent (lubricant), talc was used. As shown in Table 7, the amount of coating was selected as 40% by weight or 60% by weight with respect to a nuclear particle to be coated. As antistatic agents, Neusilin UFL2 was used.

40 g of a water insoluble macromolecule Ethocel 10FP mixed with 30 g of talc was prepared as particle mixture for coating 8. The aminoalkyl methacrylate copolymer E100 (No. 100 on) prepared in Example 5 was also used.

Examples 8-1 to 8-2 were manufactured in accordance with the formulation ratio and amount described in Table 11. Specifically, compound C and granularity controlled product of pulverized aminoalkyl methacrylate copolymer E100 (No. 100 ON fraction) were loaded into a container rotating granulator, Intensive Mixer (EL-1, Nippon Eirich) and granulated while spraying a suitable amount of aqueous 95% ethanol solution under the mixing and granulating conditions shown in Table 8 to obtain nuclear particles to be coated in a wet powder state. The nuclear particles in a wet powder state were loaded into a fluidized bed dryer (MP-01, Powrex Corp) and dried under the drying conditions shown in Table 2 to obtain nuclear particles to be coated. The nuclear particles to be coated were loaded into a container rotating granulator, Intensive Mixer (EL-1, Nippon Eirich), and coated while spraying the aqueous 95% ethanol solution described in Table 7 under the coating conditions specified in Table 8 as particle mixture for coating 8 was separated and added 12 times at 7.5 g each. When particle mixture for coating 8 was added to an amount equivalent to 40% by weight (when 60 g of particle mixture for coating was added and coated), samples were collected. The sampled component of interest-containing hollow particles in a wet powder state were loaded into a rack dryer (Perfect Oven, ESPEC Corp.) and dried for 2 hours at 60° C. to obtain the component of interest-containing hollow particles of Example 8-1. Subsequent to the sampling, the coating step was continued until 90 g of mixture for coating 8 was coated to obtain component of interest-containing hollow particles in a wet powder state. The component of interest-containing hollow particles in a wet powder state were loaded into a fluidized bed dryer (MP-01, Powrex Corp) and dried under the drying conditions shown in Table 2. After drying, Neusilin was added and mixed within the fluidized bed granulator MP-01 to obtain the component of interest-containing hollow particles of Examples 8-1 to 8-2.

TABLE 11

| | Comparative Example 8 | | Example 8-1 | | Example 8-2 | |
|---|---|---|---|---|---|---|
| | Amount | Formulation | Amount | Formulation | Amount | Formulation |
| | loaded | ratio | loaded | ratio | loaded | ratio |
| | (g) | (%) | (g) | (%) | (g) | (%) |
| Compound C | 160 | 80 | 120 | 57.1 | 120 | 49.8 |
| (ground product | | | | | | |
| of JM) | | | | | | |
| Aminoalkyl | 40 | 20 | 30 | 14.3 | 30 | 12.4 |
| methacrylate | | | | | | |
| copolymer E | | | | | | |
| (No. 100 on) | | | | | | |
| Ethocel 10FP | — | — | 40 | 19.0 | 60 | 24.9 |
| Talc | — | — | 20 | 9.5 | 30 | 12.4 |
| Neusilin UFL2 | — | — | — | — | 1 | 0.4 |
| Aqueous 95% | 45 | 22.5 | — | — | — | — |
| ethanol solution | | | | | | |
| (for nuclear | | | | | | |
| particle) | | | | | | |
| Aqueous 95% | — | — | 55 | 26.2 | 73 | 30.3 |
| ethanol solution | | | | | | |
| (for coating) | | | | | | |
| Total | 200 | 100 | 210 | 100 | 241 | 100 |

### Comparative Example 8 Manufacture of Nuclear Particles to be Coated

In Comparative Example 8, only particles that were not coated, i.e., nuclear particles to be coated, were manufactured in accordance with the formulation ratio and amount loaded described in Table 11 in the same manner as Example 8. After granulating nuclear particles to be coated in a wet powder state as in Example 8, the nuclear particles to be coated in a wet powder state were subjected to fluidized bed drying using a fluidized bed dryer (model MP-01, Powrex Corp) to obtain the nuclear particles to be coated of Comparative Example 8.

### <Test Example 8> <Dissolution Test on Hollow Particles Containing Compound C as a Component of Interest Using a Water Insoluble Macromolecule Particle as a First Macromolecule and a Stomach Soluble Macromolecule Particle as a Second Macromolecule>

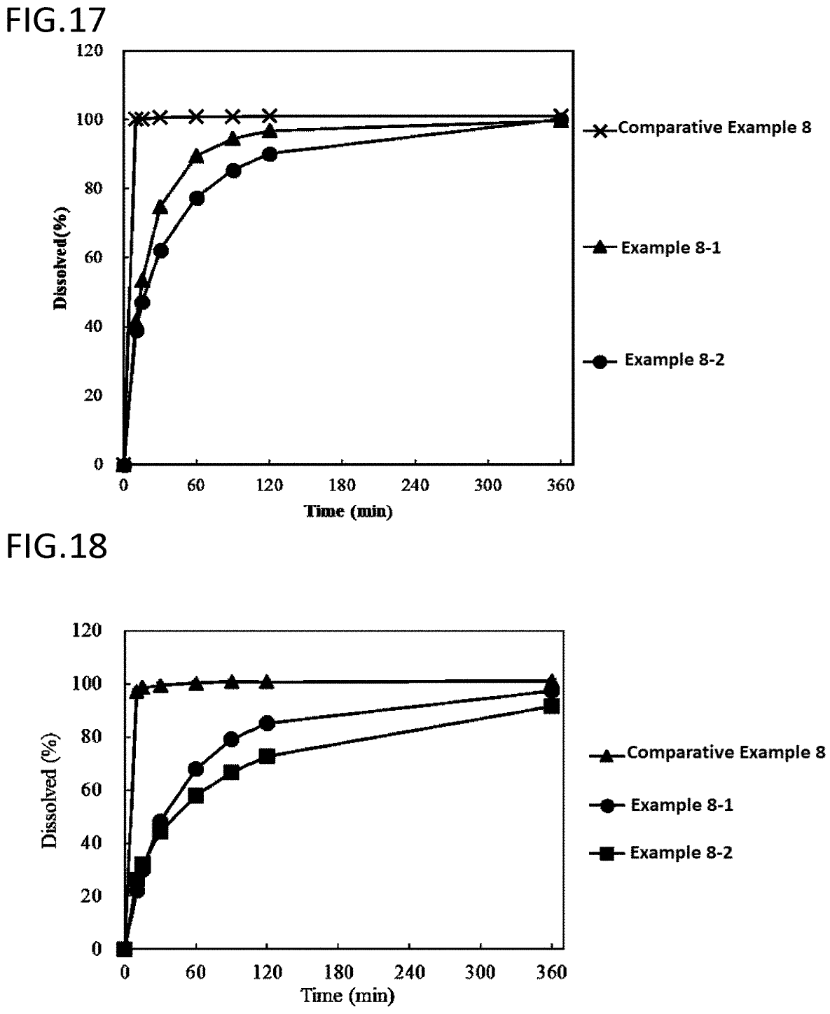

Dissolution tests were conducted using the particles manufactured in Example 8. The dissolution test conditions were the same as in Test Example 1. HPLC measurement conditions are shown below. The results are shown in FIGS. 17 and 18. FIG. 17 is the test result using 1st fluid for dissolution test, and FIG. 18 is the test result using 2nd fluid for dissolution test. Table 12 shows the coating time and the time that was required for the manufacture of the resulting particles.

<HPLC Measurement Conditions>

### HPLC: UFLC XR/Shimadzu Corporation (LC-204)

Detector: UV detector
Measurement wavelength: 272 nm
Column: Shim-Pack XR ODS (3.0 mm×75 mm, 2.2 um)
Column temperature: 40° C.
Mobile phase: water/methanol mixture (7:3)
Mobile phase flow rate: 1.0 mL/min

### Injection: 5 μL

Sample cooler temperature: 25° C.
Syringe cleansing solution: water/methanol mixture=(1:1)

Tables 12 and 13 show the ratios of 50% dissolution times before and after coating for the component of interest-containing hollow particles. An effect of suppressing the release rate was attained.

TABLE 12
50% dissolution time and ratio of 50% dissolution time with
respect to nuclear particle in 1st fluid for dissolution test

| | 50% | Ratio of 50% dissolution | Manufacturing |
|---|---|---|---|
| | dissolution | time with respect to | time |
| | time | nuclear particle | (coating (minutes)) |
| Comparative | 4.89 | — | — |
| Example 5 | | | |
| Example 5-1 | 113.70 | 23.24 | 24 |
| Example 5-2 | 893.55 | 182.64 | 45 |
| Comparative | 15.94 | — | — |
| Example 6 | | | |
| Example 6-1 | 27.53 | 1.73 | 25 |
| Example 6-2 | 421.23 | 26.42 | 45 |
| Comparative | 5.02 | — | — |
| Example 7 | | | |
| Example 7-1 | 7.36 | 1.47 | 18 |
| Example 7-2 | 38.41 | 7.65 | 36 |
| Comparative | 4.99 | — | — |
| Example 8 | | | |
| Example 8-1 | 13.55 | 2.71 | 40.5 |
| Example 8-2 | 31.72 | 6.35 | 55 |

TABLE 13
50% dissolution time and ratio of 50% dissolution time with
respect to nuclear particle in 2nd fluid for dissolution test

| | 50% | Ratio of 50% dissolution | Manufacturing |
|---|---|---|---|
| | dissolution | time with respect | time |
| | time | to nuclear particles | (coating (minutes)) |
| Comparative | 50.05 | — | Same as Table 8 |
| Example 5 | | | |
| Example 5-1 | 358.01 | 7.15 | |
| Example 5-2 | 1785.88 | 35.68 | |
| Comparative | 15.94 | — | |
| Example 6 | | | |
| Example 6-1 | 21.96 | 1.47 | |
| Example 6-2 | 390.63 | 26.13 | |
| Comparative | 5.02 | — | |
| Example 7 | | | |
| Example 7-1 | 25.93 | 4.96 | |
| Example 7-2 | 99.07 | 18.94 | |
| Comparative | 4.99 | — | |
| Example 8 | | | |
| Example 8-1 | 32.79 | 6.38 | |
| Example 8-2 | 42.72 | 8.31 | |

As disclosed above, the present disclosure is exemplified by the use of its preferred embodiments. However, it is understood that the scope of the present disclosure should be interpreted solely based on the Claims.

The present application claims priority to Japanese Patent Application No. 2018-196987 (filed on Oct. 18, 2018). The entire content thereof is incorporated herein by reference. It is also understood that any patent, any patent application, and any references cited herein should be incorporated herein by reference in the same manner as the contents are specifically described herein.

### INDUSTRIAL APPLICABILITY

The particles of the present disclosure can be utilized in solid pharmaceutical formulations.

1: A particle consisting of a shell and a hollow section, coated with a first macromolecule and a lubricant, wherein the particle comprises a second macromolecule, and comprises different properties, which are a property of the first macromolecule and a property of the second macromolecule.

2: The particle of claim 1, wherein the different properties are selected from two or more of fast release, sustained release, enteric solubility, stomach solubility, bitterness masking, and photostability.

3: The particle of claim 2, wherein the properties comprise sustained release.

4: The particle of claim 2, wherein the properties comprise enteric solubility.

5: The particle of claim 2, wherein the properties comprise stomach solubility.

6: The particle of claim 2, wherein the properties comprise bitterness masking.

7: The particle of claim 1, wherein the first macromolecule is selected from one or more of a water-soluble macromolecule, a water-insoluble macromolecule, an enteric soluble macromolecule, and a stomach soluble macromolecule.

8: The particle of claim 7, wherein the first macromolecule is a water-soluble macromolecule.

9: The particle of claim 7, wherein the first macromolecule is a water-insoluble macromolecule.

10: The particle of claim 7, wherein the first macromolecule is an enteric soluble macromolecule.

11: The particle of claim 7, wherein the first macromolecule is a stomach soluble macromolecule.

12: The manufacturing method of claim 1, wherein the lubricant is selected from one or more of magnesium aluminosilicate, talc, Red Ferric Oxide, Yellow Ferric Oxide, titanium oxide, sodium stearyl fumarate, and magnesium stearate.

13: A manufacturing method of a particle coated with a first macromolecule and a lubricant, the particle being a component of interest-containing hollow particle comprising a component of interest and a second macromolecule, the method comprising:
adding the first macromolecule and the lubricant to a nuclear particle comprising the component of interest and the second macromolecule and coating the resulting mixture by spraying a solvent that can dissolve the first macromolecule while rolling the mixture.

14: The manufacturing method of claim 13, wherein the coated particle comprises an inner core layer comprising the component of interest and the second macromolecule and a coating layer comprising the first macromolecule and the lubricant.

15: The manufacturing method of claim 13, further comprising generating the nuclear particle by mixing the component of interest and the second macromolecule.

16: The manufacturing method of claim 13, wherein a D90 value of the first macromolecule and the lubricant is 100 μm or less.

17: The manufacturing method of claim 13, wherein a mean particle size of the first macromolecule and the lubricant is 25 μm or less.

18: The manufacturing method of claim 13, wherein a D99 value of the first macromolecule and the lubricant is 150 μm or less.

19: The manufacturing method of claim 13, wherein all of the first macromolecule and the lubricant pass through a 100 mesh sieve.

20: The manufacturing method of claim 13, wherein a weight ratio of the first macromolecule to the lubricant is between 1:5 and 5:1.

21: The manufacturing method of claim 13, wherein the first macromolecule and the lubricant are 10% by weight to 100% by weight with respect to the nuclear particle.
